SEQUENCE LISTING (18 sequences)

SEQ ID NO: 1 - protein, 511 aa, Indiana vesiculovirus
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - protein, 288 aa, Human immunodeficiency virus 1
FLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 3 - protein, 288 aa, synthetic construct
FLDGIDKAQEEHEKYHSNWRAMASDFNLPPVVAKEIVASCDKCQLKGEAMHGQVDCSPGIWQLVCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 4 - protein, 401 aa, Human immunodeficiency virus 1
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAW

SEQ ID NO: 5 - protein, 401 aa, synthetic construct
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLEVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAW

SEQ ID NO: 6 - DNA, 636 bp, synthetic construct
  source 1..636: mol_type = other DNA
tggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 7 - DNA, 260 bp, synthetic construct
  source 1..260: mol_type = other DNA
tggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 8 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 9 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 10 - DNA, 627 bp, Homo sapiens
  source 1..627: mol_type = other DNA
atggcacctctcggtgaagtcggaaactacttcggagtccaggatgccgtgcccttcggcaacgtgcctgtgctgccagtggattctccagtgctgctgtctgatcacctgggtcaaagcgaggctggaggcctgcccagaggtcctgcagtgactgatctggatcacctgaagggcattctcagacgcaggcaactgtactgcagaaccggattccatctcgaaatctttccaaatggcaccattcaaggaaccagaaaggatcactctcgctttggcatcctggagttcatttccatcgctgttggcctcgtctctatcagaggcgtggacagcggtctgtacctcggaatgaacgagaagggtgagctgtatggttccgagaagctcacacaagaatgcgtgtttcgcgaacagtttgaagagaattggtacaacacctacagctccaatctgtacaagcatgtggatacaggtaggagatactatgtcgcactgaacaaagatggcactccacgcgagggtacacgcactaagaggcatcagaagttcacacatttcctgccaaggccagtggaccctgacaaggtgcctgagctctacaaggacatcctcagccagtcttga

SEQ ID NO: 11 - protein, 208 aa, Homo sapiens
MAPLGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 12 - DNA, 1158 bp, Gallus gallus
  source 1..1158: mol_type = other DNA
atgggtagcattggagcagccagcatggagttctgctttgacgtcttcaaggaactcaaagtgcatcatgcaaacgagaacatcttctactgtcccatcgcaatcatgagcgctctcgctatggtttacctgggagccaaagacagcaccaggacccagatcaacaaggtggtccgcttcgataagctgcctggtttcggagacagcatcgaggcacagtgcggaacctccgtgaacgttcattcttctctgagggacattctgaatcagatcacaaagcctaacgacgtgtatagcttctctctggcctccaggctgtacgctgaggaacgctaccctatcctccctgaatacctgcagtgtgtcaaagagctgtatagaggtggactggaacctatcaacttccaaactgcagcagaccaggcacgcgagctgatcaattcttgggtggagtctcaaactaacggaatcatcaggaacgtgctccagccctccagcgtggacagccagactgcaatggtgctggtcaacgccattgtcttcaagggactctgggagaagactttcaaggacgaggacacacaggctatgcctttcagagttactgagcaggagagcaagcctgtccagatgatgtaccagatcggtctgttcagagtggcatctatggccagcgagaagatgaagatcctggagctgcccttcgcaagcggaactatgtctatgctcgtgctgctgccagatgaggtttctggactggaacaactggagtccatcatcaactttgagaagctgacagagtggacaagctctaacgtcatggaagagagaaagatcaaggtgtacctcccaaggatgaagatggaagagaagtacaacctgactagcgtgctgatggcaatgggaatcacagatgtctttagctcttctgctaacctgtctggcatctcctccgcagagtccctgaagatttcccaggctgtccacgctgctcatgccgaaatcaatgaagcaggtagggaagtggtcggatctgcagaggctggtgttgatgctgccagcgtctccgaagagtttagagctgaccatccctttctgttctgtatcaaacatatcgccacaaatgcagtcctgttcttcggaagatgtgtgtctcct

SEQ ID NO: 13 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 14 - DNA, 4131 bp, synthetic construct
  source 1..4131: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagattttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgacctgctgaagattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggatcccccgccattaagaagggcatcctgcagacagtgaagattgtggacgagctcgtgaaagtgatgggccacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacattgtgccccagtccttcatcaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcattaagcggcagctggtggaaacccggcagatcacaaagcacgtggcacagatcctggactcccggatgaacactaagtacgacgagaacgacaaactgatccgggaagtgaaagtgatcaccctgaagtccaagctggtgtccgacttcagaaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgattacaaggtgtacgacgtgcggaagatgatcgccaagagcgagcaggaaatcggcaaggctaccgccaagtacttcttctacagcaacatcatgaactttttcaagaccgagatcacactggccaacggcgagatcagaaagcggcctctgatcgagacaaacggcgaaaccggggagatcgtgtgggataagggccgggattttgccacagtgcggaaagtgctgtccatgccccaagtgaatatcgtgaaaaagaccgaggtgcagaccggcggcttcagcaaagagtctatcctgcccaagaggaactccgacaagctgatcgccagaaagaaggattgggaccctaagaagtacggcggctttgacagccccaccgtggcctactctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagcggatgctggcttctgccggcgaactgcagaagggaaacgagctggccctgccctccaaatatgtgaacttcctgtacctggccagccactatgagaagctgaagggctcccccgaggataatgagcagaaacagctgtttgtggaacagcacaagcactacctggacgagatcatcgagcagattagcgagttctccaagcgcgtgatcctggccgatgccaacctggacaaggtgctgagcgcctacaacaagcaccgggataagcccatcagagagcaggccgagaatatcatccacctgtttaccctgaccaacctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgaccccaagaaaaagcgcaaagtgctcgagtga

SEQ ID NO: 15 - protein, 1376 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVLE

SEQ ID NO: 16 - DNA, 138 bp, synthetic construct
  source 1..138: mol_type = other DNA
tcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggag

SEQ ID NO: 17 - DNA, 205 bp, synthetic construct
  source 1..205: mol_type = other DNA
tccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacct

SEQ ID NO: 18 - DNA, 600 bp, synthetic construct
  source 1..600: mol_type = other DNA
ccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggg

(19) United States
(12) Patent Application Publication
(10) Pub. No.: US 2024/0060067 A1
(43) Pub. Date: Feb. 22, 2024

(54) TRANSIENT EXPRESSION SYSTEM FOR RNA, FOR COSMETIC USES

(71) Applicant: GEG Tech, Paris (FR)

(72) Inventor: Nicolas GRANDCHAMP, Paris (FR)

(73) Assignee: GEG TECH, Paris (FR)

(21) Appl. No.: 17/818,493

(22) Filed: Aug. 9, 2022

(51) Int. Cl. C12N 15/11; A61P 17/14; C12N 15/86

(52) U.S. Cl. CPC C12N 15/11; A61P 17/14; C12N 15/86; C12N 2740/16043

(57) ABSTRACT

A method of treating skin and/or skin appendages in a subject in need thereof, which includes administering to the subject a ribonucleic acid (RNA) molecule including, from 5′ to 3′: an RNA Booster sequence that includes or is the following ribonucleic acid sequence: mmsknkkkm, wherein: “m” indicates an adenine (a) or cytosine (c); “s” indicates a guanine (g) or a cytosine (c); “k” indicates a guanine (g) or a uracyl (u); “n” indicates any nucleotide; and a sequence of interest.

### FIELD

The present invention relates to means and methods for robust transient RNA expression.

### BACKGROUND

Transferring genetic information to a cell typically requires the design of vectors capable of delivering this genetic information to the cell. There exist two main types of vectors: non-viral vectors and viral vectors, each of which is capable of transferring information, either in the form of DNA or RNA.

When transient—rather than sustained—effects are desired, non-integrating DNA vectors or RNA vectors are excellent candidates. However, non-integrating DNA vectors can induce adverse genotoxic effects due to the non-zero probability of any DNA molecule to recombine with another DNA molecule, for instance, with the DNA genome of the host cell. RNA vectors do not exhibit this risk of genotoxicity, since RNA cannot recombine with DNA. Nonetheless, current RNA vectors are not devoid of drawbacks, in particular in terms of efficacy. For instance, non-viral RNA vectors are underperforming, due to a low degree of RNA protection against degradation, and often, a low transfection rate. Hence, the few RNA molecules which ultimately reach the cytoplasm of a cell induce only poor transgene expression.

Retroviridae, or retroviruses, is a family of RNA viruses which are capable of inserting a copy of their genome—after reverse transcription using their own reverse transcriptase enzyme (RT)—into the chromosomal DNA of the host cells that they invade. The host cells then treat the viral DNA as part of their own genome, transcribing and translating the viral genes along with their own genes. This ability of Retroviridae has made them (and more particularly viruses from the Gammaretrovirus and Lentivirus genera) a benchmark tool for therapeutic gene delivery and transfer into cells since the early 1980's. More recently, these RNA vectors have been engineered to allow transient expression of therapeutic proteins. Such engineered vectors were described in WO 2005/116225 A1, WO 2013/060819 A2 or Mock et al., 2014 (Sci Rep. 4:6409) relating to retroviral vectors with detective RT activity.

Yet, these RT-defective retroviral vectors, although offering a transient expression of therapeutic proteins from their mRNA without the need of a DNA intermediate, are not devoid of drawbacks. Indeed, they typically package only up to two copies of their RNA genome, into which the transgene of interest (e.g., a therapeutic mRNA) is inserted. In other words, one retroviral vector transducing a host cell can only deliver two copies of a transgene of interest. Given that RNAs are very labile molecules—and thus rapidly degraded in the host cell, this solution was considered somehow ineffective, unless high vector doses are repeatedly administered in order to outweigh these issues. For in vivo applications, this is not desirable. For instance, Mock et al. (2014. Sci Rep. 4:6409) came to this conclusion, stating that they “observed very weak cap-dependent translation initiation from standard lentiviral vector genomes”, and called for further improvements.

There remains thus a need for RNA vectors, capable of inducing high transient expression levels in host cells, but without requiring the administration of high loads of vector.

Here, the Inventors have identified an artificial 9-nucleotide sequence, that they have named “RNA Booster”. The Inventors have surprisingly observed that the presence of this RNA Booster upstream of a transgene highly improved transgene expression in various host cells.

### SUMMARY

The present invention relates to a method of treating skin and/or skin appendages in a subject in need thereof, comprising administering to the subject a ribonucleic acid (RNA) molecule comprising, from 5′ to 3′:
an RNA Booster sequence comprising or consisting of the following ribonucleic acid sequence: mmsknkkkm, wherein:
   “m” indicates an adenine (a) or cytosine (c);
   “s” indicates a guanine (g) or a cytosine (c);
   “k” indicates a guanine (g) or a uracyl (u);
   “n” indicates any nucleotide;
and
a sequence of interest.

In some embodiments, treating skin and/or skin appendages include one or several of inducing hair growth, preventing hair loss, inducing hair removal, hair coloring or bleaching, preventing hair graying, promoting hair thickening, promoting hair curling, prevention hair curling, promoting skin healing, preventing wrinkle formation, improving skin elasticity, inducing skin tone homogenization, and reducing sebum secretion.

In some embodiments, the RNA Booster sequence comprises or consists of a ribonucleic acid sequence mmskngkkm, preferably mmskngkgm, more preferably cmskhgkgm, even more preferably cmskwgkgm, yet even more preferably ccsuwgggm, wherein:
“m” indicates an adenine (a) or cytosine (c);
“s” indicates a guanine (g) or a cytosine (c);
“k” indicates a guanine (g) or a uracyl (u);
“h” indicates an adenine (a) or a cytosine (c) or a uracyl (u);
“w” indicates an adenine (a) or a uracyl (u);
“n” indicates any nucleotide.

In some embodiments, the RNA Booster sequence is selected from the group consisting of:
RNA Booster 9 comprising or consisting of the sequence ccguaggga;
RNA Booster 8 comprising or consisting of the sequence cccuugggc;
RNA Booster 7 comprising or consisting of the sequence cacgugugc;
RNA Booster 6 comprising or consisting of the sequence cccucgggc;
RNA Booster 5 comprising or consisting of the sequence aacuggggc;
RNA Booster 4 comprising or consisting of the sequence ccguggugc;
RNA Booster 3 comprising or consisting of the sequence cccuaggua;
RNA Booster 2 comprising or consisting of the sequence aaguuuggc; and
RNA Booster 1 comprising or consisting of the sequence cccgugugc.

In some embodiments, the RNA molecule is comprised within a non-viral vector.

In some embodiments, the RNA molecule is packaged into an RNA virus vector derived from a Group III, Group IV, Group V or Group VI RNA virus. Preferably, the RNA molecule is packaged into RNA virus vector derived from a Group VI RNA virus. More preferably, the RNA molecule is packaged into a Retroviridae vector.

In some embodiments, the Retroviridae vector is an Orthoretrovirinae or a Spumaretrovirinae. Preferably, the Retroviridae vector is an Orthoretrovirinae. More preferably, the Retroviridae vector is selected from the group consisting of human immunodeficiency viruses (HIV), simian immunodeficiency viruses (SIV), feline immunodeficiency virus (FIV), bovine immunodeficiency virus (BIV), puma lentivirus (PLV), equine infectious anemia virus (EIAV), caprine arthritis encephalitis virus (CAEV), Visna-maedi virus, Jembrana disease virus, avian sarcoma leukosis virus (ASLV), Rous sarcoma virus (RSV), avian myeloblastosis virus (AMV), mouse mammary tumor virus (MMTV), Jaagsiekte sheep retrovirus (JSRV), enzootic nasal tumor viruses (ENTV), simian retroviruses (SRV), Mason-Pfizer monkey virus (M-PMV), human T-lymphotropic viruses (HTLV), simian T-lymphotropic viruses (STLV), bovine leukemia virus (BLV), Walleye dermal sarcoma virus (WDSV), Walleye epidermal hyperplasia viruses (WEHV), murine leukemia viruses (MLV), Abelson murine leukemia virus (AMLV), Friend virus (FV), feline leukemia virus (FeLV), koala retrovirus (KoRV), xenotropic murine leukemia virus-related virus (XMRV), chick syncytial virus (CSV), murine sarcoma viruses (MSV), feline sarcoma viruses (FSV), Gibbon ape leukemia virus (GaLV), guinea pig type-C oncovirus, porcine type-C oncovirus, reticuloendotheliosis virus, Trager duck spleen necrosis virus, viper retrovirus, and Woolly monkey sarcoma virus.

In some embodiments, the Retroviridae vector is a lentiviral vector. Preferably, the Retroviridae vector is a lentiviral vector selected from the group consisting of human immunodeficiency virus-1 (HIV-1) and HIV-2. More preferably, the Retroviridae vector is HIV-1.

In some embodiments, the Group VI RNA virus vector (preferably the Retroviridae vector) is reverse transcriptase (RT)-defective. Preferably, the Group VI RNA virus vector (preferably the Retroviridae vector) does not comprise a gene encoding a reverse transcriptase or wherein the retroviral vector comprises a gene encoding a mutated reverse transcriptase with abolished reverse transcription activity.

In some embodiments where the RNA molecule is packaged into an RNA virus vector, the RNA molecule further comprises one or several of:
a 5′ long terminal repeat (LTR),
a packaging sequence,
a Rev-response element sequence,
a post-transcriptional regulation element sequence, and
a3′LTR.

In some embodiments, the sequence of interest is selected from the group consisting of sequences encoding the glia-activating factor (FGF9), the hepatocyte growth factor (HGF), the platelet-derived growth factor (PDGF), the fibroblast growth factor 5 (FGF5), the fibroblast growth factor 10 (FGF10), the transforming growth factor β1 (TGFβ1), the transforming growth factor α (TGFα), the keratinocyte growth factor (KGF), the insulin-like growth factor 1 (IGF-1), the insulin-like growth factor 2 (IGF-2), the insulin-like growth factor-binding protein 5 (IGFBP5), the vascular endothelial growth factor (VEGF), the acidic fibroblast growth factor (aFGF), the basic fibroblast growth factor (bFGF), the epidermal growth factor (EGF), the L-dopachrome tautomerase (TRP2), noggin (NOG), protaetiamycine, thioredoxin (TRX), superoxide dismutase (SOD1), the stem cell factor (SCF), and the human growth hormone (hGH).

In some embodiments, the sequence of interest encodes the glia-activating factor (FGF9); preferably the non-therapeutic method is for inducing hair growth and/or promoting skin healing.

In some embodiments, the RNA molecule is to be administered to the subject in need thereof topically, preferably cutaneously or transdermally.

In some embodiments, the RNA molecule is to be administered to the subject in need thereof transdermally, preferably after collagen induction therapy (CIT) or through a transdermal patch.

### BRIEF DESCRIPTION OF THE DRAWINGS

FIGS. 1A and 1B show the expression of GFP in 293T cells transduced with an RNA vector derived from lentivirus expressing GFP, and comprising one of RNA Booster 1 to RNA Booster 9, or without RNA Booster (0 RNA Booster). 1A is a histogram showing the relative efficacy of GFP expression in 293T cells. The relative expression based on the level of expression after transduction with an RNA vector derived from lentivirus without RNA Booster is shown. 1B is a histogram showing the transientness of GFP expression in 293T cells. Results are shown as a percentage of 293T cells expressing GFP at day 3 (D3) and day 7 (D7) post-transduction.

FIG. 2 is a histogram showing the percentage of GFP-positive human PBMC after transduction with an integrating lentiviral vector expressing GFP [Integrating LV-GFP], or an RNA vector derived from lentivirus with a mutated reverse transcriptase (D110E substitution) expressing GFP and comprising RNA Booster 8 at two different doses, as indicated [LV-RNA Booster 8/GFP], versus negative control without transduction [0 vector].

FIG. 3 is a histogram showing the percentage of GFP-positive human dendritic cells after transduction with an integrating lentiviral vector expressing GFP [Integrating LV-GFP], a non-integrating lentiviral vector expressing GFP [Non-integrating LV-GFP], or an RNA vector derived from lentivirus with a mutated reverse transcriptase (D110E substitution) expressing GFP and comprising RNA Booster 8 [LV-RNA Booster 8/GFP], versus negative control without transduction [[Ø vector].

FIG. 4 is an histogram showing the percentage of GFP-positive human hematopoietic stem cells after transduction with an integrating lentiviral vector expressing GFP [Integrating LV-GFP], or a non-integrating lentiviral vector expressing GFP [Non-integrating LV-GFP], or an RNA vector derived from lentivirus with a mutated reverse transcriptase (D110E substitution) expressing GFP and comprising RNA Booster 8 at two different doses, as indicated [LV-RNA Booster 8/GFP], versus negative control without transduction [0 vector].

FIG. 5 is a histogram showing the results of a cell proliferation assay of human hair follicle dermal papilla cells transduced or not with different doses (as indicated) of an RNA vector derived from lentivirus with a mutated reverse transcriptase (D110E substitution) expressing FGF9 and comprising RNA Booster 8 [LV-RNA Booster 8/FGF9], and cultured in presence or absence of cortisol and/or VEGF (as indicated).

FIG. 6 is a histogram showing the quantification of immunoglobulin G (IgG) against ovalbumin (OVA) in C57BL/6J mice after prime/boost immunization with unadjuvanted ovalbumin [Pos Unadj OVA], adjuvanted OVA [Pos Adj OVA (Alun)], or with an RNA vector derived from lentivirus with a mutated reverse transcriptase (D110E substitution) expressing ovalbumin and comprising RNA Booster 8 [LV-RNA-OVA], or with a non-integrating lentiviral vector expressing ovalbumin [LV-DNA-OVA] versus negative control [Neg C]. Prime and boost injections were performed by various routes as indicated [prime/boost]: subcutaneously [SC], intramuscularly [IM], intranasally [IN], intraperitoneally [IP].

FIG. 7 is an histogram showing the percentage of GFP-positive HeLa cells constitutively expressing GFP after a co-transduction with an RNA vector derived from lentivirus with a mutated reverse transcriptase (D110E substitution) expressing Cas9 and comprising RNA Booster 8 [LV-RNA Booster 8/Cas9] and a non-integrating lentiviral vector expressing a guide RNA targeting the GFP [Non-integrating LV-gRNA], versus a co-transduction with a non-integrating lentiviral vector expressing Cas9 [Non-integrating LV-Cas9] and a non-integrating lentiviral vector expressing a guide RNA targeting GFP [Non-integrating LV-gRNA], versus negative control without transduction [[Ø vector].

### DETAILED DESCRIPTION

A first object of the invention is a ribonucleic acid (RNA) molecule.

According to the invention, the RNA molecule comprises, from 5′ to 3′:
an RNA Booster sequence characterized by a sequence mmsknkkkm, and
a sequence of interest.

According to the invention, the RNA molecule comprises an RNA Booster sequence. The term “RNA Booster” as used herein refers to an artificial 9-nucleotide sequence, which comprises or consists of a sequence mmsknkkkm, wherein:
“m” indicates an adenine (a) or a cytosine (c);
“s” indicates a guanine (g) or a cytosine (c);
“k” indicates a guanine (g) or a thymine/uracyl (t/u);
“n” indicates any nucleotide.

In some embodiments, the RNA Booster sequence comprises or consists or a sequence mmskngkkm, wherein:
“m” indicates an adenine (a) or a cytosine (c);
“s” indicates a guanine (g) or a cytosine (c);
“k” indicates a guanine (g) or a thymine/uracyl (t/u);
“n” indicates any nucleotide.

In some embodiments, the RNA Booster sequence comprises or consists or a sequence mmskngkgm, wherein:
“m” indicates an adenine (a) or a cytosine (c);
“s” indicates a guanine (g) or a cytosine (c);
“k” indicates a guanine (g) or a thymine/uracyl (t/u);
“n” indicates any nucleotide.

In some embodiments, the RNA Booster sequence comprises or consists or a sequence cmskhgkgm, wherein:
“m” indicates an adenine (a) or a cytosine (c);
“s” indicates a guanine (g) or a cytosine (c);
“k” indicates a guanine (g) or a thymine/uracyl (t/u);
“h” indicates an adenine (a) or a cytosine (c) or a thymine/uracyl (t/u).

In some embodiments, the RNA Booster sequence comprises or consists or a sequence cmskwgkgm, wherein:
“m” indicates an adenine (a) or a cytosine (c);
“s” indicates a guanine (g) or a cytosine (c);
“w” indicates an adenine (a) or a thymine/uracyl (t/u);
“h” indicates an adenine (a) or a cytosine (c) or a thymine/uracyl (t/u).

In some embodiments, the RNA Booster sequence comprises or consists or a sequence ccsuwgggm, wherein:
“s” indicates a guanine (g) or a cytosine (c);
“w” indicates an adenine (a) or a thymine/uracyl (t/u);
“m” indicates an adenine (a) or a cytosine (c).

Exemplary RNA Booster sequences include, but are not limited to:
RNA Booster 1: cccgugugc
RNA Booster 2: aaguuuggc
RNA Booster 3: cccuaggua
RNA Booster 4: ccguggugc
RNA Booster 5: aacuggggc
RNA Booster 6: cccucgggc
RNA Booster 7: cacgugugc
RNA Booster 8: cccuugggc
RNA Booster 9: ccguaggga

In order of preference, the RNA molecule comprises an RNA Booster sequence selected from the group comprising or consisting of RNA Booster 9, RNA Booster 8, RNA Booster 7, RNA Booster 6, RNA Booster 5, RNA Booster 4, RNA Booster 3, RNA Booster 2, and RNA Booster 1.

In some embodiments, the RNA molecule comprises an RNA Booster sequence selected from the group comprising or consisting of RNA Booster 9, RNA Booster 8, RNA Booster 7, RNA Booster 6, RNA Booster 5, RNA Booster 4, RNA Booster 3, and RNA Booster 2.

In some embodiments, the RNA molecule comprises an RNA Booster sequence selected from the group comprising or consisting of RNA Booster 9, RNA Booster 8, RNA Booster 7, RNA Booster 6, RNA Booster 5, RNA Booster 4, and RNA Booster 3.

In some embodiments, the RNA molecule comprises an RNA Booster sequence selected from the group comprising or consisting of RNA Booster 9, RNA Booster 8, RNA Booster 7, RNA Booster 6, RNA Booster 5, and RNA Booster 4.

In some embodiments, the RNA molecule comprises an RNA Booster sequence selected from the group comprising or consisting of RNA Booster 9, RNA Booster 8, RNA Booster 7, RNA Booster 6, and RNA Booster 5.

In some embodiments, the RNA molecule comprises an RNA Booster sequence selected from the group comprising or consisting of RNA Booster 9, RNA Booster 8, RNA Booster 7, and RNA Booster 6.

In some embodiments, the RNA molecule comprises an RNA Booster sequence selected from the group comprising or consisting of RNA Booster 9, RNA Booster 8, and RNA Booster 7.

In some embodiments, the RNA molecule comprises an RNA Booster sequence selected from the group comprising or consisting of RNA Booster 9, and RNA Booster 8.

In some embodiments, the RNA molecule comprises RNA Booster 9. In some embodiments, the RNA molecule comprises RNA Booster 8.

According to the invention, the RNA Booster is located in 5′ of a sequence of interest.

In some embodiments, the RNA Booster is located from 50 ribonucleotides (50 nt) to 1000 ribonucleotides (1 knt) upstream of a sequence of interest; such as, e.g., about 50±25 nt, 100±25 nt, 150±25 nt, 200±25 nt, 250±25 nt, 300±25 nt, 350±25 nt, 400±25 nt, 450±25 nt, 500±25 nt, 550±25 nt, 600±25 nt, 650±25 nt, 700±25 nt, 750±25 nt, 800±25 nt, 850±25 nt, 900±25 nt, 950±25 nt or 1±0.025 knt upstream of a sequence of interest. In some preferred embodiments, the RNA Booster is located about 500±25 nt upstream of a sequence of interest.

According to the invention, the RNA molecule comprises a sequence of interest.

The term “sequence of interest”, sometimes also referred to as “transgene (of interest)”, refers to any nucleic acid sequence encoding a product of interest. The product of interest may be a protein or a fragment thereof; in this case, the sequence of interest is said to be a coding nucleic acid sequence. However, the term also encompasses non-coding nucleic acid sequences, i.e., nucleic acid sequences that do not encode a protein or a fragment thereof, but rather express an “RNA gene” (or “non-coding RNA”), such as, e.g., a transfer RNA, a ribosomal RNA, a small RNA, a long non-coding RNA, etc. The context will indicate whether the term “sequence of interest” refers to a DNA or an RNA sequence.

The sequence of interest can be a sequence encoding a peptide or protein (e.g., without limitation, an enzyme, a transcription factor, a growth factor, a trophic factor, a hormone, a cytokine, an antibody, an antigen, a receptor, an immune regulator, a differentiation factor, a suicide protein, a cell-cycle modifying protein, an anti-proliferative protein, an angiogenic factor, an anti-angiogenic factor, a genome editor, a nuclease, a recombinase, a transposase, a neurotransmitter, and a reporter, including any precursor thereof, as well as fusion proteins). In this case, the sequence of interest may typically be (or be derived from) an mRNA, a cDNA, a gDNA, a synthetic nucleic acid, or any combinations thereof.

The sequence of interest can alternatively be a sequence of a non-coding RNA.

Examples of non-coding RNAs include, but are not limited to, transfer RNAs (tRNAs), ribosomal RNAs (rRNAs), small nuclear RNAs (snRNAs), small nucleolar RNAs (snoRNAs), SmY RNAs, small Cajal body-specific RNAs (scaRNAs), guide RNAs (gRNAs), Y RNAs, telomerase RNA component (TERC), spliced leader RNAs (SL RNAs), catalytic RNAs (i.e., ribozymes; such as, e.g., ribonuclease P, ribonuclease MRP, and the like), antisense RNAs (aRNAs), cis-natural antisense transcript (cis-NAT), CRISPR RNAs (crRNAs), long non-coding RNAs (lncRNAs), microRNAs (miRNAs), piwi-interacting RNAs (piRNAs), small interfering RNAs (siRNAs), short hairpin RNAs (shRNAs), trans-acting siRNAs (tasiRNAs), repeat-associated siRNAs (rasiRNAs), 7SK RNAs (7SK), enhancer RNAs (eRNAs), and RNA aptamers.

Examples of sequences of interest include any nucleic acid sequence encoding a molecule of therapeutic interest, such as any nucleic acid sequence encoding a peptide or protein, or a non-coding RNA, that is lacking, deficient and/or non-functional in a subject affected with a disease or condition.

Some specific, non-limiting, examples of sequences of interest include nucleic acid sequence encoding a CRISPR element (such as, e.g., Cas9, Cas12 or a gRNA), a zinc finger protein, a transcription activator-like effector nuclease (TALEN), a meganuclease, a spike protein of an enveloped virus (such as the spike protein of a Coronaviridae, e.g., of SARS-CoV-2; of an Orthomyxoviridae, e.g., of an Influenza virus; or of a Retroviridae), a fibroblast growth factor (such as, e.g., the glia-activating factor FGF9), a vascular endothelial growth factor (VEGF, including its isoforms, e.g., VEGF₁₂₁, VEGF_{121b}, VEGF₁₄₅, VEGF₁₆₅, VEGF_{165b}, VEGF₁₈₉, VEGF₂₀₆), a neutrophic factor (such as, e.g., a brain-derived neurotrophic factor BDNF, a ciliary neurotrophic factor CNTF, or a glial cell line-derived neurotrophic factor GDNF), an antibody or an antigen-binding fragment thereof, a chimeric antigen receptor, a fluorescent reporter (such as, e.g., GFP, RFP or YFP), luciferase, an alternative oxidase (AOX), a recombinase (such as, e.g., Cre or Flp), a glutathione peroxidase, a myoblast determination protein 1 (MyoD), a superoxide dismutase (SOD), a transcription factor (such as, e.g., Oct3/4, c-Myc, Kruppel-like factor 4 KLF4, or Sox2), a tumor antigen (such as, e.g., p53), an immune checkpoint (such as, e.g., PD-1 or PD-L1), a microbial rhodopsin (such as, e.g., a halorhodopsin, in particular the halorhodopsin from Natronomonas [NpHR] and more particularly the engineered halorhodopsin from Natronomonas [eNpHR3.0], or a channelrhodopsin, in particular ChR2), a MAP kinase (such as, e.g., MAPK10, in particular the MAPK10 from Leishmania donovani [LmjMAPK10]), and an interleukin (such as, e.g., IL-7 or IL-2).

In some preferred embodiments, the sequence of interest is an antigen, and the RNA molecule of the invention is particularly suitable for vaccination purposes.

In some preferred embodiments, the sequence of interest is a genome editor, and the RNA molecule of the invention is particularly suitable for genome editing purposes.

In some preferred embodiments, the sequence of interest is FGF9, and the RNA molecule of the invention is particularly suitable for cosmetic purposes.

In the context of the invention, the expression of the sequence of interest is transient (i.e., not permanent or sustained). The duration and amount of expression may be increased, e.g., by inserting a post-transcriptional regulatory element in 3′ (i.e., downstream) of the sequence of interest.

“Post-transcriptional regulation elements” are cis-acting RNA sequences that can increase the accumulation of cytoplasmic mRNA by promoting mRNA exportation from the nucleus to the cytoplasm, enhancing 3′-end processing and stability.

Examples of post-transcriptional regulatory element include, but are not limited to, the post-transcriptional regulatory element of Woodchuck hepatitis virus (WPRE) and the post-transcriptional regulatory element of hepatitis B virus (HPRE).

As demonstrated in the Examples, the duration of expression of the sequence of interest in a cell, in particular in the presence of a post-transcriptional regulatory element, may be at most 7 days.

In some embodiments, the RNA molecule is an mRNA molecule.

In some embodiments, the mRNA molecule is comprised within a non-viral vector. The present invention encompasses thus a non-viral vector comprising the RNA molecule described herein.

According to these embodiments, the mRNA molecule may further comprise:
upstream of the RNA Booster sequence: a 5′-UTR sequence comprising a cap structure, and
downstream of the sequence of interest, a 3′-UTR and a 3′-polyA.

In some embodiments, the cap structure is a 5′-terminal m⁷G(5′)ppp(5′)G. In some embodiments, the cap structure may be an analog of m⁷G(5′)ppp(5′)G, such as, e.g., 3′-O-Me-m⁷G(5′)ppp(5′)G (called anti-reverse cap analog or ARCA), m⁷G(5′)ppp(5′)A, G(5′)ppp(5′)G or G(5′)ppp(5′)A.

In some embodiments, the non-viral vector 1) has efficient encapsulation and protection on mRNAs from nuclease-based degradation upon administration to a subject; 2) prolongs the mRNAs half-life by preventing rapid clearance by a subject's kidney and phagocytosis by the subject's liver or spleen upon administration; 3) enhances targeted tissue/organ penetration and accumulation; 4) facilitates targeted cell internalization; 5) avoids lysosomal degradation during intracellular trafficking pathway; and/or 6) enhances the release of mRNAs in a subject's cell cytoplasm to exert gene effects.

Typical examples of non-viral RNA vectors include, but are not limited to, liposomes, exosomes, lipid nanoparticles, polypeptide nanoparticles, stable nucleic acid lipid particle (SNALP), and cationic lipoplexes.

Alternatively, the RNA molecule may be packaged into or otherwise comprised within a viral vector. The present invention encompasses thus a viral vector comprising the RNA molecule described herein.

In some embodiments, the RNA molecule is packaged into or otherwise comprised within an RNA virus vector, i.e., an engineered viral particle derived from an RNA virus. By “engineered viral particle”, it is implied that (i) at least one exogenous nucleic acid sequence is introduced into the genome of the RNA virus, and/or (ii) at least one endogenous gene from the RNA virus is be mutated or deleted, either partially or totally.

RNA viruses are viruses that have ribonucleic acid as their genetic material. RNA viruses can be classified into four groups according to the Baltimore classification system:
Group III (double-stranded RNA viruses), including the following families: Birnaviridae, Chrysoviridae, Cystoviridae, Hypoviridae, Partitiviridae, Reoviridae, Totiviridae and Endornavirus. Exemplary genera of double-stranded RNA viruses that can infect humans include, without limitation, Rotavirus and Coltivirus.
Group IV (positive-sense single-stranded RNA viruses), including the following families: Arteriviridae, Coronaviridae, Roniviridae, Astroviridae, Barnaviridae, Bromoviridae, Caliciviridae, Closteroviridae, Comoviridae, Dicistroviridae, Flaviviridae, Flexiviridae, Hepeviridae, Leviviridae, Luteoviridae, Marnaviridae, Narnaviridae, Nodaviridae, Picornaviridae, Potyviridae, Sequiviridae, Tetraviridae, Togaviridae, Tombusviridae, Tymoviridae, Virgaviridae, Benyvirus, Cheravirus, Idaeovirus, Machlomovirus, Ourmiavirus, Sadwavirus Sobemovirus and Umbravirus. Exemplary species of positive-sense single-stranded RNA viruses that can infect humans include, without limitation, hepatitis C virus, yellow fever virus, West Nile virus, dengue virus, Zika virus, Chikungunya virus, rubella virus, MERS, SARS, and SARS-CoV-2.
Group V (negative-sense single-stranded RNA viruses), including the following families: Qinviridae, Aspiviridae, Chuviridae, Bornaviridae, Filoviridae, Mymonaviridae, Nyamiviridae, Paramyxoviridae, Pneumoviridae, Rhabdoviridae, Sunviridae, Yueviridae, Arenaviridae, Cruliviridae, Feraviridae, Fimoviridae, Hantaviridae, Jonviridae, Nairoviridae, Peribunyaviridae, Phasmaviridae, Phenuiviridae, Tospoviridae, Amnoonviridae and Orthomyxoviridae. Exemplary species of negative-sense single-stranded RNA viruses that can infect humans include, without limitation, Ebola virus, Lassa virus, Marburg virus, measles virus, rabies virus, mumps virus, Influenza viruses, and respiratory syncytial virus.
Group VI (negative-strand single-stranded RNA-reverse transcriptase viruses), including the following families: Metaviridae, Pseudoviridae and Retroviridae.

Exemplary species of negative-strand single-stranded RNA-reverse transcriptase viruses that can infect humans include, without limitation, human immunodeficiency virus (HIV), and human T-cell leukemia-lymphoma virus (HTLV).

In some embodiments, the RNA molecule is packaged into or otherwise comprised within an RNA virus vector derived from a Group III, Group IV, Group V or Group VI RNA virus.

In some preferred embodiments, the RNA molecule is packaged into or otherwise comprised within an RNA virus vector derived from a Group VI RNA virus.

In order to be packaged into or otherwise comprised within an RNA virus vector, the RNA molecule may comprise a packaging sequence.

By “packaging sequence”, it is referred to a stem-loop structured cis-acting nucleic acid sequence, which regulates the process of packaging inside a viral capsid. This packaging sequence may be referred in the art to as “packaging signal” denoted “T”, or “encapsidation signal” denoted “E”. Packaging sequences may be of viral origin (i.e., wild-type viral packaging sequences) or may be synthetic.

Viral packaging sequences include bacteriophage packaging sequences (such as, e.g., DNA phage packaging sequences and RNA phage packaging sequences) and eukaryotic virus packaging sequences (such as, e.g., DNA virus packaging sequences and RNA virus packaging sequences).

When of viral origin, the packaging sequence may be:
(i) a wild-type packaging sequence from an RNA virus; or preferably
(ii) a wild-type packaging sequence from an RNA virus of the same group as the one of the RNA virus into which the RNA molecule is to be packaged into, or more preferably
(iii) a wild-type packaging sequence from an RNA virus of the same family as the one of the RNA virus into which the RNA molecule is to be packaged into, or even more preferably
(iv) a wild-type packaging sequence from an RNA virus of the same genus as the one of the RNA virus into which the RNA molecule is to be packaged into, or yet even more preferably
(v) the wild-type packaging sequence from the RNA virus into which the RNA molecule is to be packaged into.

In some embodiments, the packaging sequence may be a modified packaging sequence which shares more than 70%, 75%, 80%, 85%, 90%, 95% of sequence identity or even more with the wild-type packaging sequence of (i), (ii), (iii), (iv) or (v) above, while retaining its ability to trigger the process of packaging inside a viral capsid.

Examples of packaging sequences include, but are not limited to, the psi (F) packaging signal of HIV or SIV; the core encapsidation signal from Gammaretrovirus; the epsilon (F) encapsidation signal from HBV, and the encapsidation signal from BLV.

In some preferred embodiments, the RNA molecule is packaged into or otherwise comprised within an RNA virus vector derived from a Group VI RNA virus of the Retroviridae family. The present invention encompasses thus a retroviral vector comprising the RNA molecule described herein.

Retroviruses are enveloped viruses from the Retroviridae family. They package two identical single-stranded ribonucleic acid (RNA) molecules of typically 7 to 10 kb in length, forming their genome. The genome of retroviruses typically comprises gag, pol and env genes flanked by two long terminal repeat (LTRs) sequences. Each of these genes encodes for numerous peptides, which are initially expressed in the form of a single precursor polypeptide. The gag gene encodes for the internal structure proteins (matrix, capsid and nucleocapsid); the pol gene encodes for retroviral enzymes reverse transcriptase, integrase and protease; and the env gene encodes for viral envelope glycoprotein. The genome of retroviruses can further contain cis-acting elements, e.g., elements responsible for exporting out of the nucleus the unspliced viral genomic RNA which will be packaged, such as a Rev-response element (RRE) sequence. The 5′ and 3′ LTRs serve to promote transcription and also serve as a polyadenylation sequence of the viral RNAs. Sequences necessary for the initiation of reverse transcription of the genome and for the encapsidation of viral RNA in particles (psi [F] packaging element) are typically adjacent to the 5′ LTR. If the sequences necessary for encapsidation are absent from the viral genome, the genomic RNA will not be actively packaged; it can, however, be passively packaged although with a lower yield. The genome of more complex retroviruses may comprise additional genes encoding accessory and/or regulatory proteins such as src, sag, tax, vif, vpr, vpx, vpu, nef, tat, rev, tmx, tas and/or bet. For example, the HIV-1 genome contains 7 accessory genes: vif, vpr, vpx, vpu, nef, tat and rev.

The Retroviridae family is subdivided into two subfamilies: Orthoretrovirinae and Spumaretrovirinae. In some embodiments, the retroviral vector is (or is derived from) an Orthoretrovirinae or a Spumaretrovirinae. In some preferred embodiments, the retroviral vector is (or is derived from) an Orthoretrovirinae.

The Orthoretrovirinae subfamily is subdivided into six genera: Alpharetrovirus, Betaretrovirus, Deltaretrovirus, Epsilonretrovirus, Gammaretrovirus, and Lentivirus.

Exemplary species of Alpharetrovirus include, but are not limited to, avian sarcoma leukosis virus (ASLV), Rous sarcoma virus (RSV), and avian myeloblastosis virus (AMV).

Exemplary species of Betaretrovirus include, but are not limited to, mouse mammary tumor virus (MMTV), Jaagsiekte sheep retrovirus (JSRV), enzootic nasal tumor viruses (ENTV; including ENTV-1 and ENTV-2), simian retroviruses (SRV; including SRV-1 and SRV-2), and Mason-Pfizer monkey virus (M-PMV; formerly known as SRV-3).

Exemplary species of Deltaretrovirus include, but are not limited to, human T-lymphotropic viruses (HTLV; including HTLV-1, HTLV-2, HTLV-3 and HTLV-4), simian T-lymphotropic viruses (STLV; including STLV-1, STLV-2, STLV-3, and STLV-4), and bovine leukemia virus (BLV).

Exemplary species of Epsilonretrovirus include, but are not limited to, Walleye dermal sarcoma virus (WDSV), and Walleye epidermal hyperplasia viruses (WEHV; including WEHV-1 and WEHV-2).

Exemplary species of Gammaretrovirus include, but are not limited to, murine leukemia viruses (MLV), Abelson murine leukemia virus (AMLV), Friend virus (FV), feline leukemia virus (FeLV), koala retrovirus (KoRV), xenotropic murine leukemia virus-related virus (XMRV), chick syncytial virus (CSV), murine sarcoma viruses (MSV; including Finkel-Biskis-Jinkins murine sarcoma virus, Harvey murine sarcoma virus, Kirsten murine sarcoma virus and Moloney murine sarcoma virus), feline sarcoma viruses (FSV; including Gardner-Arnstein feline sarcoma virus, Hardy-Zuckerman feline sarcoma virus and Snyder-Theilen feline sarcoma virus), Gibbon ape leukemia virus (GaLV), guinea pig type-C oncovirus, porcine type-C oncovirus, reticuloendotheliosis virus, Trager duck spleen necrosis virus, viper retrovirus, and Woolly monkey sarcoma virus.

Exemplary species of Lentivirus include, but are not limited to, human immunodeficiency viruses (HIV; including HIV-1 and HIV-2), simian immunodeficiency viruses (SIV), feline immunodeficiency virus (FIV), bovine immunodeficiency virus (BIV), puma lentivirus (PLV), equine infectious anemia virus (EIAV), caprine arthritis encephalitis virus (CAEV), Visna-maedi virus, and Jembrana disease virus.

In some embodiments, the retroviral vector is (or is derived from) an Alpharetrovirus, a Betaretrovirus, a Deltaretrovirus, an Epsilonretrovirus, a Gammaretrovirus or a Lentivirus.

In some preferred embodiments, the retroviral vector is (or is derived from) a Lentivirus. In some preferred embodiments, the retroviral vector is (or is derived from) a Lentivirus selected from the group comprising or consisting of human immunodeficiency viruses (HIV; including HIV-1 and HIV-2), simian immunodeficiency viruses (SIV), feline immunodeficiency virus (FIV), bovine immunodeficiency virus (BIV), puma lentivirus (PLV), equine infectious anemia virus (EIAV), caprine arthritis encephalitis virus (CAEV), Visna-maedi virus (VMV), and Jembrana disease virus (JDV).

In some preferred embodiments, the retroviral vector is (or is derived from) a human immunodeficiency virus, such as HIV-1 and HIV-2; more preferably HIV-1.

In some embodiments, a retroviral vector as described herein packages two RNA molecules, each comprising an RNA Booster sequence and a sequence of interest, as described above. According to this embodiment, the two RNA molecules may comprise the same sequence of interest, or different sequences of interest.

Also contemplated herein is therefore a retroviral vector comprising a recombinant ribonucleic genome, itself comprising, from 5′ to 3′:
an RNA Booster sequence, and
a sequence of interest.

In some embodiments, the retroviral vector comprises a recombinant ribonucleic genome, itself comprising, from 5′ to 3′:
a packaging sequence,
an RNA Booster sequence, and
a sequence of interest.

In some embodiments, the retroviral vector comprises a recombinant ribonucleic genome, itself comprising, from 5′ to 3′:
a 5′ LTR,
a packaging sequence,
an RNA Booster sequence,
a sequence of interest, and
a 3′ LTR.

In some embodiments, the retroviral vector comprises a recombinant ribonucleic genome, itself comprising, from 5′ to 3′:
a 5′ LTR,
a packaging sequence,
a Rev-response element,
an RNA Booster sequence,
a sequence of interest, and
a 3′ LTR.

In some embodiments, the retroviral vector comprises a recombinant ribonucleic genome, itself comprising, from 5′ to 3′:
a 5′ LTR,
a packaging sequence,
a Rev-response element,
an RNA Booster sequence,
a sequence of interest,
a post-transcriptional regulatory element, and
a 3′ LTR.

In some embodiments, the retroviral vector is reverse transcriptase-defective.

In other words, the gene encoding the reverse transcriptase may be mutated or deleted, so that the reverse transcription process is altered and cannot be completed, i.e., cannot give rise to a full-length retroviral double-stranded DNA intermediate molecule upon infection of a target cell, and consequently, cannot generate a proviral vector genome. This inability, in the context of the invention, can result either from (i) an absence of the reverse transcriptase gene in the retroviral vector, or (ii) at least one mutation in the reverse transcriptase gene in the retroviral vector.

Examples of mutations in the reverse transcriptase gene to abolish reverse transcription activity are known in the art. Reference is made, inter alia, to WO 2013/060819 A2, from page 15 line 31 to page 42 line 14, which selected passage is hereby incorporated by reference. For instance, a HIV-1 reverse transcriptase with SEQ ID NO: 4 may comprise a D110E substitution resulting in an abolished reverse transcriptase activity.

In some embodiments, the retroviral vector might further be integrase-defective.

In other words, the gene encoding the integrase might be mutated or deleted, so that the integration process is altered and cannot be completed, i.e., a proviral vector genome cannot be integrated into the genome of a target cell. This inability can result either from (i) an absence of the integrase gene in the retroviral vector, or (ii) at least one mutation in the integrase gene in the retroviral vector.

In some embodiments, the RNA molecule, in particular when packaged into or otherwise comprised within an RNA virus vector, may further comprise one or several elements, in particular one or several of long terminal repeats (LTR), including a 5′ LTR and a 3′ LTR; a Rev-response element (RRE) sequence; and a post-transcriptional regulation element sequence.

“Long-terminal repeats” are sequences of several hundred base pairs long. In RNA viruses, their genome is flanked by LTRs (a 5′ LTR and a 3′ LTR), typically having identical sequences.

The “Rev-response element” is a highly structured RNA segment interacting with the Rev protein, allowing the viral genome to be exported to the cytoplasm for downstream processing, including virion packaging. Rev-response elements are typically characteristic of lentiviruses, but other RNA viruses of Group VI comprise similar systems, such as the Rem-response element in Betaretroviruses, the Rex-response element in Deltaretroviruses, or the constitutive transport element (CTE). These are also encompassed when mentioning Rev-response element herein, even if not explicitly cited.

In some embodiments, the RNA molecule comprises a 5′ LTR, located in 5′ of the packaging sequence (in other words, before the packaging sequence).

In some embodiments, the RNA molecule comprises a Rev-response element (RRE) sequence, located in 3′ of the packaging sequence but in 5′ of the RNA Booster sequence (in other words, between the packaging sequence and the RNA Booster sequence).

In some embodiments, the RNA molecule comprises a post-transcriptional regulation element sequence, located in 3′ of the sequence of interest (in other words, after the sequence of interest).

In some embodiments, the RNA molecule comprises a 3′ LTR, located in 3′ of the sequence of interest (in other words, after the sequence of interest). When a post-transcriptional regulation element sequence is present, the 3′ LTR is located in 3′ of this post-transcriptional regulation element sequence.

In some embodiments, the RNA molecule may thus comprise, from 5′ to 3′:
optionally, a 5′ LTR,
compulsorily, a packaging sequence,
optionally, a Rev-response element,
compulsorily, an RNA Booster sequence,
compulsorily, a sequence of interest,
optionally, a post-transcriptional regulatory element, and
optionally, a 3′ LTR.

A second object of the invention is a composition comprising, consisting of, or consisting essentially of, the RNA molecule described above. In particular, an object of the invention is a composition comprising, consisting of, or consisting essentially of, the non-viral vector described above, or the viral vector, in particular the retroviral vector, described above.

In some embodiment, the composition is a pharmaceutical composition and further comprises at least one pharmaceutically acceptable excipient or carrier.

As used herein, the term “consist essentially of” and any declension thereof, with reference to a composition, means that the RNA molecule (or the vector comprising said RNA molecule), is the only one therapeutic agent or agent with a biological activity within said composition or pharmaceutical composition.

The terms “pharmaceutically acceptable excipient” and “pharmaceutically acceptable carrier” include any and all solvents, dispersion media, coatings, antibacterial and antifungal agents, isotonic and absorption delaying agents, and the like, that are physiologically compatible. The excipient or carrier does not produce any adverse, allergic or other untoward reaction when administered to a subject, preferably to a human. A pharmaceutically acceptable excipient or carrier is typically a non-toxic solid, semi-solid or liquid filler, diluent, encapsulating material or formulation auxiliary of any type. For human administration, preparations should meet sterility, pyrogenicity, and general safety and purity standards as required by regulatory offices, such as, for example, the FDA (US Food and Drug Administration) or EMA (European Medicines Agency).

Pharmaceutically acceptable excipients or carriers that may be used in the composition or pharmaceutical composition include, but are not limited to, ion exchangers, alumina, aluminum stearate, lecithin, serum proteins (such as human serum albumin), buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

A third object of the invention relates to the various uses and applications of the RNA molecule, in particular of the non-viral vector or viral vector, in particular the retroviral vector, or of the composition, as described above.

These uses and applications are particularly suitable in eukaryotes, whether in vitro, ex vivo or in vivo. In some embodiments, the eukaryote is an animal, preferably a mammal, even more preferably a human.

Encompassed in these uses and applications is a method, in particular an in vitro or ex vivo method, of transiently expressing a sequence of interest in a cell, which method comprises contacting or otherwise transfecting or transducing the cell with the RNA molecule, or with the non-viral vector or viral vector, in particular the retroviral vector, as described above.

Also encompassed in these uses and applications is the RNA molecule, or the non-viral vector or viral vector, in particular the retroviral vector, or of the composition, for use in therapy, as a drug or as a medicament.

Also encompassed in these uses and applications is the RNA molecule, or the non-viral vector or viral vector, in particular the retroviral vector, or the composition, for use in preventing or treating a disease in a subject in need thereof; or a method of preventing or treating a disease, comprising administering to a subject in need thereof the RNA molecule, or the non-viral vector or viral vector, in particular the retroviral vector, or the composition.

In some embodiments, the disease is selected from the group consisting of cancer, infectious diseases, neuromuscular diseases, ocular diseases, blood diseases, cardiovascular diseases, skin diseases, and neurodegenerative diseases.

In some embodiments, the disease is cancer.

Examples of cancers include those listed in the 11^{th} revision of the International Statistical Classification of Diseases and Related Health Problems (ICD), under chapter 02 “Neoplasms”.

Further examples of cancers include, but are not limited to, recurrent, metastatic or multi-drug resistant cancer.

Further examples of cancers include, but are not limited to, adenofibroma, adenoma, agnogenic myeloid metaplasia, AIDS-related malignancies, ameloblastoma, anal cancer, angiofollicular mediastinal lymph node hyperplasia, angiokeratoma, angiolymphoid hyperplasia with eosinophilia, angiomatosis, anhidrotic ectodermal dysplasia, anterofacial dysplasia, apocrine metaplasia, apudoma, asphyxiating thoracic dysplasia, astrocytoma (including, e.g., cerebellar astrocytoma and cerebral astrocytoma), atriodigital dysplasia, atypical melanocytic hyperplasia, atypical metaplasia, autoparenchymatous metaplasia, basal cell hyperplasia, benign giant lymph node hyperplasia, bile duct cancer (including, e.g., extrahepatic bile duct cancer), bladder cancer, bone cancer, brain tumor (including, e.g., brain stem glioma, cerebellar astrocytoma glioma, malignant glioma, supratentorial primitive neuroectodermal tumors, visual pathway and hypothalamic glioma, ependymoma, medulloblastoma, gestational trophoblastic tumor glioma, and paraganglioma), branchionia, female breast cancer, male breast cancer, bronchial adenomas/carcinoids, bronchopulmonary dysplasia, cancer growths of epithelial cells, pre-cancerous growths of epithelial cells, metastatic growths of epithelial cells, carcinoid heart disease, carcinoid tumor (including, e.g., gastrointestinal carcinoid tumor), carcinoma (including, e.g., carcinoma of unknown primary origin, adrenocortical carcinoma, islet cells carcinoma, adeno carcinoma, adeoncortical carcinoma, basal cell carcinoma, basosquamous carcinoma, bronchiolar carcinoma, Brown-Pearce carcinoma, cystadenocarcinoma, ductal carcinoma, hepatocarcinoma, Krebs carcinoma, papillary carcinoma, oat cell carcinoma, small cell lung carcinoma, non-small cell lung carcinoma, squamous cell carcinoma, transitional cell carcinoma, Walker carcinoma, Merkel cell carcinoma, and skin carcinoma), cementoma, cementum hyperplasia, cerebral dysplasia, cervical cancer, cervical dysplasia, cholangioma, cholesteatoma, chondroblastoma, chondroectodermal dysplasia, chordoma, choristoma, chrondroma, cleidocranial dysplasia, colon cancer, colorectal cancer, local metastasized colorectal cancer, congenital adrenal hyperplasia, congenital ectodermal dysplasia, congenital sebaceous hyperplasia, connective tissue metaplasia, craniocarpotarsal dysplasia, craniodiaphysial dysplasia, craniometaphysial dysplasia, craniopharyngioma, cylindroma, cystadenoma, cystic hyperplasia (including, e.g., cystic hyperplasia of the breast), cystosarconia phyllodes, dentin dysplasia, denture hyperplasia, diaphysial dysplasia, ductal hyperplasia, dysgenninoma, dysplasia epiphysialis hemimelia, dysplasia epiphysialis multiplex, dysplasia epiphysialis punctate, ectodermal dysplasia, Ehrlich tumor, enamel dysplasia, encephaloophthalmic dysplasia, endometrial cancer (including, e.g., ependymoma and endometrial hyperplasia), ependymoma, epithelial cancer, epithelial dysplasia, epithelial metaplasia, esophageal cancer, Ewing's family of tumors (including, e.g., Ewing's sarcoma), extrahepatic bile duct cancer, eye cancer (including, e.g., intraocular melanoma and retinoblastoma), faciodigitogenital dysplasia, familial fibrous dysplasia of jaws, familial white folded dysplasia, fibroma, fibromuscular dysplasia, fibromuscular hyperplasia, fibrous dysplasia of bone, florid osseous dysplasia, focal epithelial hyperplasia, gall bladder cancer, ganglioneuroma, gastric cancer (including, e.g., stomach cancer), gastrointestinal carcinoid tumor, gastrointestinal tract cancer, gastrointestinal tumors, Gaucher's disease, germ cell tumors (including, e.g., extracranial germ cell tumors, extragonadal germ cell tumors, and ovarian germ cell tumors), giant cell tumor, gingival hyperplasia, glioblastoma, glomangioma, granulosa cell tumor, gynandroblastoma, hamartoma, head and neck cancer, hemangioendothelioma, hemangioma, hemangiopericytoma, hepatocellular cancer, hepatoma, hereditary renal-retinal dysplasia, hidrotic ectodermal dysplasia, histiocytonia, histiocytosis, hypergammaglobulinemia, hypohidrotic ectodermal dysplasia, hypopharyngeal cancer, inflammatory fibrous hyperplasia, inflammatory papillary hyperplasia, intestinal cancers, intestinal metaplasia, intestinal polyps, intraocular melanoma, intravascular papillary endothelial hyperplasia, kidney cancer, laryngeal cancer, leiomyoma, leukemia (including, e.g., acute lymphoblastic leukemia, acute lymphocytic leukemia, acute myeloid leukemia, acute myelogenous leukemia, acute hairy cell leukemia, acute B-cell leukemia, acute T-cell leukemia, acute HTLV leukemia, chronic lymphoblastic leukemia, chronic lymphocytic leukemia, chronic myeloid leukemia, chronic myelogenous leukemia, chronic hairy cell leukemia, chronic B-cell leukemia, chronic T-cell leukemia, and chronic HTLV leukemia), Leydig cell tumor, lip and oral cavity cancer, lipoma, liver cancer, lung cancer (including, e.g., small cell lung cancer and non-small cell lung cancer), lymphangiomyoma, lymphaugioma, lymphoma (including, e.g., AIDS-related lymphoma, central nervous system lymphoma, primary central nervous system lymphoma, Hodgkin's lymphoma, non-Hodgkin's lymphoma, Hodgkin's lymphoma during pregnancy, non-Hodgkin's lymphoma during pregnancy, mast cell lymphoma, B-cell lymphoma, adenolymphoma, Burkitt's lymphoma, cutaneous T-cell lymphoma, large cell lymphoma, and small cell lymphoma), lymphopenic thymic dysplasia, lymphoproliferative disorders, macroglobulinemia (including, e.g., Waldenstrom's macroglobulinemia), malignant carcinoid syndrome, malignant mesothelioma, malignant thymoma, mammary dysplasia, mandibulofacial dysplasia, medulloblastoma, meningioma, mesenchymoma, mesonephroma, mesothelioma (including, e.g., malignant mesothelioma), metaphysial dysplasia, metaplastic anemia, metaplastic ossification, metaplastic polyps, metastatic squamous neck cancer (including, e.g., metastatic squamous neck cancer with occult primary), Mondini dysplasia, monostotic fibrous dysplasia, mucoepithelial dysplasia, multiple endocrine neoplasia syndrome, multiple epiphysial dysplasia, multiple myeloma/plasma cell neoplasm, mycosis fungoides, myelodysplastic syndrome, myeloid metaplasia, myeloproliferative disorders, chronic myeloproliferative disorders, myoblastoma, myoma, myxoma, nasal cavity and paranasal sinus cancer, nasopharyngeal cancer, prostatic neoplasm, colon neoplasm, abdomen neoplasm, bone neoplasm, breast neoplasm, digestive system neoplasm, liver neoplasm, pancreas neoplasm, peritoneum neoplasm, endocrine glands neoplasm (including, e.g., adrenal neoplasm, parathyroid neoplasm, pituitary neoplasm, testicles neoplasm, ovary neoplasm, thymus neoplasm, and thyroid neoplasm), eye neoplasm, head and neck neoplasm, nervous system neoplasm (including, e.g., central nervous system neoplasm and peripheral nervous system neoplasm), lymphatic system neoplasm, pelvic neoplasm, skin neoplasm, soft tissue neoplasm, spleen neoplasm, thoracic neoplasm, urogenital tract neoplasm, neurilemmoma, neuroblastoma, neuroepithelioma, neurofibroma, neurofibromatosis, neuroma, nodular hyperplasia of prostate, nodular regenerative hyperplasia, oculoauriculovertebral dysplasia, oculodentodigital dysplasia, oculovertebral dysplasia, odontogenic dysplasia, odontoma, opthalmomandibulomelic dysplasia, oropharyngeal cancer, osteoma, ovarian cancer (including, e.g., ovarian epithelial cancer and ovarian low malignant potential tumor), pancreatic cancer (including, e.g., islet cell pancreatic cancer and exocrine pancreatic cancer), papilloma, paraganglioma, nonchromaffin paraganglioma, paranasal sinus and nasal cavity cancer, paraproteinemias, parathyroid cancer, periapical cemental dysplasia, pheochromocytoma (including, e.g., penile cancer), pineal and supratentorial primitive neuroectodermal tumors, pinealoma, pituitary tumor, plasma cell neoplasm/multiple myeloma, plasmacytoma, pleuropulmonary blastoma, polyostotic fibrous dysplasia, polyps, pregnancy cancer, pre-neoplastic disorders (including, e.g., benign dysproliferative disorders such as benign tumors, fibrocystic conditions, tissue hypertrophy, intestinal polyps, colon polyps, esophageal dysplasia, leukoplakia, keratoses, Bowen's disease, Farmer's skin, solar cheilitis, and solar keratosis), primary hepatocellular cancer, primary liver cancer, primary myeloid metaplasia, prostate cancer, pseudoachondroplastic spondyloepiphysial dysplasia, pseudoepitheliomatous hyperplasia, purpura, rectal cancer, renal cancer (including, e.g., kidney cancer, renal pelvis, ureter cancer, transitional cell cancer of the renal pelvis and ureter), reticuloendotheliosis, retinal dysplasia, retinoblastoma, salivary gland cancer, sarcomas (including, e.g., uterine sarcoma, soft tissue sarcoma, carcinosarcoma, chondrosarcoma, fibrosarcoma, hemangiosarcoma, Kaposi's sarcoma, leiomyosarcoma, liposarcoma, lymphangiosarcoma, myosarcoma, myxosarcoma, rhabdosarcoma, sarcoidosis sarcoma, osteosarcoma, Ewing sarcoma, malignant fibrous histiocytoma of bone, and clear cell sarcoma of tendon sheaths), sclerosing angioma, secondary myeloid metaplasia, senile sebaceous hyperplasia, septooptic dysplasia, Sertoli cell tumor, Sezary syndrome, skin cancer (including, e.g., melanoma skin cancer and non-melanoma skin cancer), small intestine cancer, spondyloepiphysial dysplasia, squamous metaplasia (including, e.g., squamous metaplasia of amnion), stomach cancer, supratentorial primitive neuroectodermal and pineal tumors, supratentorial primitive neuroectodermal tumors, symptomatic myeloid metaplasia, teratoma, testicular cancer, theca cell tumor, thymoma (including, e.g., malignant thymoma), thyroid cancer, trophoblastic tumors (including, e.g., gestational trophoblastic tumors), ureter cancer, urethral cancer, uterine cancer, vaginal cancer, ventriculoradial dysplasia, verrucous hyperplasia, vulvar cancer, Waldenstrom's macroglobulinemia, and Wilms' tumor.

In some embodiments, the disease is an infectious disease.

Examples of infectious diseases include those listed in the 11^{th} revision of the International Statistical Classification of Diseases and Related Health Problems (ICD), under chapter 01 “Certain infectious or parasitic diseases”.

Further examples of infectious diseases include, but are not limited to, bacterial infections, viral infections, fungal infections, parasitic infections, ectoparasitic infections, and the like.

In some embodiments, the disease is a neuromuscular disease.

Examples of neuromuscular diseases include, but are not limited to, acid maltase deficiency, amyotrophic lateral sclerosis, Andersen-Tawil syndrome, Becker muscular dystrophy, Becker myotonia congenita, Bethlem myopathy, bulbospinal muscular atrophy, carnitine deficiency, carnitine palmityl transferase deficiency, central core disease, centronuclear myopathy, Charcot-Marie-Tooth disease, congenital muscular dystrophy, congenital myasthenic syndromes, congenital myotonic dystrophy, Cori disease, Debrancher enzyme deficiency, Dejerine-Sottas disease, dermatomyositis, distal muscular dystrophy, Duchenne muscular dystrophy, dystrophia myotonica, Emery-Dreifuss muscular dystrophy, endocrine myopathies, Eulenberg disease, facioscapulohumeral muscular dystrophy, tibial distal myopathy, Friedreich's ataxia, Fukuyuma congenital muscular dystrophy, glycogenosis type 2, glycogenosis type 3, glycogenosis type 5, glycogenosis type 7, glycogenosis type 9, glycogenosis type 10, glycogenosis type 11, Gowers-Laing distal myopathy, hereditary inclusion-body myositis, hypothyroid myopathy, inclusion-body myositis, inherited myopathies, integrin-deficient congenital muscular dystrophy, spinal-bulbar muscular atrophy, spinal muscular atrophy, lactate dehydrogenase deficiency, Lambert-Eaton myasthenic syndrome, McArdei disease, merosin-deficient congenital muscular dystrophy, metabolic diseases of muscle, mitochondrial myopathy, Miyoshi distal myopathy, motor neuron disease, muscle-eye-brain disease, myasthenia gravis, myoadenylate deaminase deficiency, myofibrillar myopathy, myophosphorylase deficiency, myotonia congenital, myotonic muscular dystrophy, myotubular myopathy, nemaline myopathy, Nonaka distal myopathy, oculopharyngeal muscular dystrophy, paramyotonia congenital, Pearson syndrome, periodic paralysis, phosphofructokinase deficiency, phosphoglycerate kinase deficiency, phosphoglycerate mutase deficiency, phosphorylase deficiency, polymyositis, Pompe disease, progressive external ophthalmoplegia, spinal muscular atrophy, Ullrich congenital muscular dystrophy, Weiander distal myopathy, and ZASP-related myopathy.

In some embodiments, the disease is an ocular disease.

Examples of ocular diseases include those listed in the 11^{th} revision of the International Statistical Classification of Diseases and Related Health Problems (ICD), under chapter 09 “Diseases of the visual system”.

Further examples of ocular diseases include, but are not limited to, age-related macular degeneration (AMD, e.g., wet AMD, dry AMD, intermediate AMD, advanced AMD, and geographic atrophy (GA)), macular degeneration, macular edema, diabetic macular edema (DME, e.g., focal, non-center DME and diffuse, center-involved DME), retinopathy, diabetic retinopathy (DR, e.g., proliferative DR (PDR), non-proliferative DR (NPDR), and high-altitude DR), other ischemia-related retinopathies, ROP, retinal vein occlusion (RVO, e.g., central (CRVO) and branched (BRVO) forms), choroidal neovascularization (CNV, e.g., myopic CNV), corneal neovascularization, diseases associated with corneal neovascularization, retinal neovascularization, diseases associated with retinal/choroidal neovascularization, pathologic myopia, von Hippel-Lindau disease, histoplasmosis of the eye, familial exudative vitreoretinopathy (FEVR), Coats' disease, Nome disease, osteoporosis-pseudoglioma syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilledema, retinitis, conjunctivitis (e.g., infectious conjunctivitis and non-infectious (e.g., allergic) conjunctivitis), Leber congenital amaurosis, uveitis (e.g., infectious or non-infectious uveitis), choroiditis (e.g., multifocal choroiditis), ocular histoplasmosis, blepharitis, dry eye, traumatic eye injury, and Sjogren's disease.

In some embodiments, the disease is a blood disease.

Examples of blood diseases include those listed in the 11^{th} revision of the International Statistical Classification of Diseases and Related Health Problems (ICD), under chapter 03 “Diseases of the blood or blood-forming organs”.

Further examples of blood diseases include, but are not limited to, acute myeloid leukemia, acute promyelocytic leukemia, acute lymphoblastic leukemia, chronic myelogenous leukemia, myelodysplastic syndromes, anemia, methaemoglobinaemia, hemophilia, non-thrombocytopenic purpura, thrombocytosis, and thrombocytopenia.

In some embodiments, the disease is a cardiovascular disease.

Examples of cardiovascular diseases include those listed in the 11^{th} revision of the International Statistical Classification of Diseases and Related Health Problems (ICD), under chapter 11 “Diseases of the circulatory system”.

Further examples of cardiovascular diseases include, but are not limited to, aneurysm, angina, arrhythmia, atherosclerosis, cardiomyopathy, stroke, cerebrovascular disease, congenital heart disease, congestive heart failure, myocarditis, valve disease coronary, artery disease dilated, cardiomyopathy, diastolic dysfunction, endocarditis, hypertension, hypertrophic cardiomyopathy, mitral valve prolapse, myocardial infarction, and venous thromboembolism.

In some embodiments, the disease is a skin disease.

Examples of skin diseases include those listed in the 11^{th} revision of the International Statistical Classification of Diseases and Related Health Problems (ICD), under chapter 14 “Diseases of the skin”.

Further examples of skin diseases include, but are not limited to, atopic dermatitis, hand dermatitis, contact dermatitis, allergic contact dermatitis, irritant contact dermatitis, neurodermatitis, perioral dermatitis, stasis dermatitis, dyshidrotic eczema, xerotic dermatitis, nummalar dermatitis, seborrheic dermatitis, eyelid dermatitis, diaper dermatitis, dermatomyositis, lichen planus, lichen sclerosis, alopecia areata, vitiligo, rosacea, epidermolysis bullosa, keratosis pilaris, pityriasis alba, pemphigus, vulvovaginitis, acne, chronic spontaneous urticaria, chronic idiopathic urticaria, chronic physical urticaria, Vogt-Koyanagi-Harada disease, Sutton nevus, post inflammatory hypopigmentation, senile leukoderma, chemical/drug-induced leukoderma, cutaneous lupus erythematosus, discoid lupus, palmoplantar pustulosis, pemphigoid, sweet's syndrome, hidradenitis suppurativa, psoriasis, plaque psoriasis, pustular psoriasis, nail psoriasis, flexural psoriasis, guttate psoriasis, psoriatic arthritis, erythrodermic psoriasis, or inverse psoriasis.

In some embodiments, the disease is a neurodegenerative disease.

Examples of neurodegenerative diseases include, but are not limited to, Alzheimer's disease, Parkinson's disease, amyotrophic lateral sclerosis (ALS), frontotemporal dementia, spinocerebellar ataxia (SCA) type 1, SCA type 2, SCA type 6, SCA type 7, SCA type 17, Machado-Joseph disease/SCA type 3 (MJD/SCA3), Huntington's disease, dentatorubral pallidoluysian atrophy (DRPLA), spinal and bulbar muscular atrophy (SBMA), prion disease, and motor neuron disease.

Also encompassed in these uses and applications is the RNA molecule, or the non-viral vector or viral vector, in particular the retroviral vector, or the composition, for use in skin and/or skin appendages (such as hair, nails or skin glands) treatment; or a method of treating skin and/or skin appendages (such as hair, nails or skin glands) in a subject in need thereof against, comprising administering to the subject in need thereof the RNA molecule, or the non-viral vector or viral vector, in particular the retroviral vector, or the composition.

In some embodiments, these uses and methods are preferably non-therapeutic uses and methods, i.e., a cosmetic use or method.

Examples of treatments of skin and/or skin appendages, in particular of non-therapeutic treatments, include for instance any or several of induction of hair growth, prevention of hair loss, induction of hair removal, hair coloring or bleaching, prevention of hair graying, promotion of hair thickening, promotion of or prevention of hair curling, promotion of skin healing, prevention of wrinkle formation, improvement of skin elasticity, induction of skin tone homogenization, and reduction of sebum secretion.

As exemplified herein, inducing hair growth and/or promoting skin healing can be achieved using the RNA molecule, in particular the retroviral vector, of the invention, in particular wherein the sequence of interest encodes the glia-activating factor (aka FGF9).

However, the skilled artisan will readily recognize that other sequences of interest can be used for cosmetic purposes, including, for instance, sequences coding for growth factors.

In some embodiments, other sequences of interest that can be used for cosmetic purposes include, but are not limited to, sequences encoding the hepatocyte growth factor (HGF), the platelet-derived growth factor (PDGF), the fibroblast growth factor 5 (FGF5) (including FGF5-short [FGF5s] and FGF5-long [FGF51]), the fibroblast growth factor 10 (FGF10), the transforming growth factor β1 (TGFβ1), the transforming growth factor α (TGFα), the keratinocyte growth factor (KGF), the insulin-like growth factor 1 (IGF-1), the insulin-like growth factor 2 (IGF-2), the insulin-like growth factor-binding protein 5 (IGFBP5), the vascular endothelial growth factor (VEGF), the acidic fibroblast growth factor (aFGF), the basic fibroblast growth factor (bFGF), the epidermal growth factor (EGF), the L-dopachrome tautomerase (TRP2), noggin (NOG), protaetiamycine, thioredoxin (TRX), superoxide dismutase (SOD1), the stem cell factor (SCF), and the human growth hormone (hGH).

In some embodiments, the RNA molecule, the non-viral vector or viral vector, in particular the retroviral vector, or the composition, is to be administered topically, in particular cutaneously or transdermally.

In some embodiments, the RNA molecule, the non-viral vector or viral vector, in particular the retroviral vector, or the composition, is to be administered topically after collagen induction therapy (CIT).

As used herein, the term “collagen induction therapy”, also known as “microneedling”, “dermarolling”, or “skin needling”, refers to a cosmetic procedure which involves repeatedly puncturing the skin of a subject with microneedles (i.e., needles having a length typically ranging from about 100 μm to 1 000 μm).

Also encompassed herein is therefore a kit comprising a microneedling device and the RNA molecule, the non-viral vector or viral vector, in particular the retroviral vector, or the composition of the invention.

Alternatively, the RNA molecule, the non-viral vector or viral vector, in particular the retroviral vector, or the composition, is to be administered topically though application onto the skin of a transdermal patch, in particular of a microneedle transdermal patch, comprising said RNA molecule, non-viral vector or viral vector, in particular the retroviral vector, or the composition of the invention.

Also encompassed herein is therefore a transdermal patch, in particular a microneedle transdermal patch, comprising the RNA molecule, the non-viral vector or viral vector, in particular the retroviral vector, or the composition of the invention.

Also encompassed in these uses and applications is the RNA molecule, or the non-viral vector or viral vector, in particular the retroviral vector, or the composition, for use in vaccinating a subject in need thereof against an infectious disease or against cancer; or a method of vaccinating a subject in need thereof against an infectious disease or against cancer, comprising administering to the subject in need thereof the RNA molecule, or the non-viral vector or viral vector, in particular the retroviral vector, or the composition.

As exemplified herein, vaccinating a subject can be efficiently achieved using the RNA molecule, in particular the retroviral vector, of the invention.

As will be readily understood by the skilled artisan, the sequence of interest in this instance should encode an antigen against which an immunization is desired.

As used herein, the terrn “anfigen”, also terrned “immunogen”, refers to any substance that induces a state of sensitivity and/orimmune responsiveness after any Iatent period (normally, days to weeks in humans) and that reacts in a demonstrabie way with antibodies and/or immune cells of the sensitized subject in vivo or in vitro.

Examples of antigens include, without limitation, pathogen-related antigens (such as, e.g., antigens of viruses, fungi or bacteria, or immunogenie molecules derived from them), self-antigens (such as, e.g_cellular antigens including cells containing normal transplantation antigens and/or turmor-related antigens, RR-Rh antigens, and antigens characteristic of, or specific to particular cells or tissues or body fluids), and allergen-related antigens (such as, e.g., hose associated with environrnental allergens, including grasses, pollens, molds, dust, insects, dander, venoms, and the like; occupational allergens, including latex, dander, urethanes, epoxy resins, and the like; food, including shellfish, peanuts, eggs, milk products, and the like; and drugs, including antibiotics, anesthetics, and the like).

Suitable examples of pathogen-related antigens include, but are not limited to, antigens derived from vaccinia, avipox virus, turkey influenza virus, bovine leukemia virus, feline leukemia virus, avian influenza, chicken pneumovirosis virus, canine parvovirus, equine influenza, FHV, Newcastle disease virus (NDV), Chicken/Pennsylvania/1/83 influenza virus, infectious bronchitis virus, Dengue virus, measles virus, Rubella virus, pseudorabies, Epstein-Barr virus, HIV, SIV, EHV, BHV, HCMV, MERS, SARS, SARS-CoV-2, Hantaan, C. tetani, mumps, Morbillivirus, Herpes Simplex virus type 1, Herpes Simplex virus type 2, Human cytomegalovirus, hepatitis A virus, hepatitis B virus, hepatitis C virus, hepatitis E virus, respiratory syncytial virus, human papilloma virus, Influenza virus, Salmonella, Neisseria, Borrelia, Chlamydia, Bordetella, Plasmodium, Toxoplasma, Cryptococcus, Streptococcus, Staphylococcus, Haemophilus, Diptheria, Tetanus, Pertussis, Escherichia, Candida, Aspergillus, Entamoeba, Giardia, and Trypanasoma.

Suitable examples of self-antigens include, but are not limited to, lupus autoantigen, Smith, Ro, La, U1-RNP, fibrillin, nuclear antigens, histones, glycoprotein gp70, ribosomal proteins, pyruvate dehydrogenase, dehydrolipoamide acetyltransferase (PCD-E2), hair follicle antigens, human tropomyosin isoform 5 (hTM5), proinsulin, insulin, IA2, GAD65, collagen type II, human cartilage gp 39 (HCgp39), gp130-RAPS, dnaJp1, citrullinated proteins and peptides (including citrullinated type II collagen, citrullinated vimentin and citrullinated fibrinogen), myelin basic protein, proteolipid protein (PLP), myelin oligodendrocyte glycoprotein (MOG), thyroid stimulating factor receptor (TSH-R), acetylcholine receptor (AchR), gliadin, PLP, glucose-6-phosphate isomerase, thyroglobulin, various tRNA synthetases, proteinase-3, and myeloperoxidase, and the like, including fragments thereof.

Suitable ex anples of iunor-related axtigens include, but are not limited to, MART-1/Melan-A, gp100, dipeptidyl peptidase IV (DPPIV), adenosine deaminase-binding protein (ADAbp), cyclophilin b, colorectal associated antigen (CRC)-C017-1A/GA733, carcinoembryonic antigen (CEA) and its immunogenic epitopes CAP-1 and CAP-2, etv6, amll, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A1l, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family (e.g. MUC1, MUC16, etc.), HER2/neu, p2iras, RCAS1, alpha-fetoprotein, E-cadherin, alpha-catenin, beta-catenin and gamma-catenin, pl20ctn, gp100.sup.Pmelll7, PRAME, NY-ESO-1, cdc27, adenomatous polyposis coli protein (APC), fodrin, connexin 37, Ig-idiotype, p15, gp75, GM2 and GD2 gangliosides, Smad family of cancer antigens brain glycogen phosphorylase, SSX-1, SSX-2 (HOM-MEL-40), SSX-1, SSX-4, SSX-5, SCP-1 and CT-7, and c-erbB-2 and viral antigens such as the HPV-16 and HPV-18 E6 and E7 antigens and the EBV-encoded nuclear antigen (EBNA)-1, and the like, including fragments thereof. Further examples of tumor-related antigens are described in, e.g., Li et al., 2004. Cancer Immunol Immunother. 53(3):139-43; Novellino et al., 2005. Cancer Immunol Immunother. 54(3):187-20; which are herein incorporated by reference in their entirety.

In some embodiments, the RNA molecule, the non-viral vector or viral vector, in particular the retroviral vector, or the composition, can be administered to a subject in need thereof once, twice, or more.

When administered twice or more, the first administration is typically referred to as “priming step”, and the subsequent administration(s) are referred to as “boosting step”.

In some embodiments, the boosting step can be carried out once, twice, three times, four times or more.

In some embodiments, the period of time between the priming step and the boosting step and/or between each iteration of the “boosting step” ranges from about 1 day to about 6 months, preferably from about 1 week to about 3 months, more preferably from about 2 weeks to about 1 month.

In one embodiment, the period of time between the priming step and the boosting step and/or between each iteration of the “boosting step” is about 1 day, 2 days, 3 days, 4 days, 5 days, 6 days, 1 week, 2 weeks, 3 weeks, 1 month, 2 months, 3 months, 4 months, 5 months, or 6 months.

In some embodiments, administration to the subject may be carried out by systemic injection. Examples of systemic injections include, but are not limited to, intravenous (iv), subcutaneous (sc), intramuscular (im), intradermal (id), intraperitoneal (ip), and intranasal (in) injection.

In case of prime/boosting administration, each injection may be carried out via the same route, or by different route. By way of non-limiting example, the priming step can be carried out by intramuscular injection and the boosting step can be carried out by intranasal injection; or the priming step can be carried out by intraperitoneal injection and boosting step by intranasal injection. Alternatively, both the priming and boosting steps can be carried out by intraperitoneal injection.

Also encompassed in these uses and applications is the RNA molecule, or the non-viral vector or viral vector, in particular the retroviral vector, or the composition, for use in genome engineering; or a method of genome engineering, comprising administering to a subject in need thereof the RNA molecule, or the non-viral vector or viral vector, in particular the retroviral vector, or the composition.

Such genome engineering applications are useful, in particular in the field of bioproduction, or in methods of therapeutic treatment such as cell therapy or gene therapy.

These uses and methods may be in vivo or in vitro.

They may also be ex vivo, wherein the RNA molecule, or the non-viral vector or viral vector, in particular the retroviral vector, or the composition, is contacted ex vivo with a cell or a population of cells from a subject, and optionally the cell or population of cells is then administered back to said subject.

As will be readily understood by the skilled artisan, the sequence of interest in this instance should encode a genome editor.

Examples of genome editors include, but are not limited to, CRISPR-associated proteins (Cas), zinc finger nucleases (ZNFs), transcription activator-like effector nucleases (TALEN), and site-specific recombinases (such as, e.g., a Cre recombinase or flippase [Flp]).

Examples of CRISPR-associated proteins include, without limitation, class 2 Cas proteins such as, e.g., Cas9, Cas12 (including Cas12a (or Cpf1), Cas12b (or C2c1), Cas12c (or C2c3), Cas12d (or CasY), Cas12e (or CasX), Cas12f (or Cas14 or C2c10), Cas12g, Cas12h, Cas12i, and Cas12k (or C2c5)), and Cas13 (including Cas13a (or C2c2), Cas13b, Cas13c, and Cas13d).

In some embodiments, genome editors may require at least one RNA molecule, in particular in the case of CRISPR-associated proteins, which RNA molecule is capable of interacting with the genome editor and targeting it to a locus of interest in a cell's genome. Said RNA molecule may be known in the art as guide RNA (gRNA). Alternatively, two RNA molecules (a CRISPR RNA named crRNA and a trans-activating CRISPR RNA named tracrRNA) may be used in lieu of a single gRNA.

In these embodiments, the uses and methods may thus comprise contacting the cell or administering to the subject such gRNA, or a mix of crRNA and tracrRNA, before, concomitantly with or after, the RNA molecule, or the non-viral vector or viral vector, in particular the retroviral vector, or the composition.

In some embodiments, genome editors may further require at least one exogenous nucleic acid, in particular at least one exogenous DNA, which is to be inserted in the genome of a target cell.

The term “exogenous” refers here to a molecule that is not naturally present in a cell, but can be introduced into the cell by one or more genetic, biochemical or other methods. Natural presence in the cell may be determined with respect to the particular developmental stage and environmental conditions of the cell. For instance, a molecule that is present only in a cell during embryonic development is exogenous with respect to a cell in an adult subject. Similarly, a molecule induced by heat shock of a cell is exogenous with respect to a non-heat-shocked cell.

This exogenous nucleic acid may comprise, for instance, the sequence of a gene of interest to be inserted in the genome of a target cell. This gene of interest may be, for instance, a functional version of a malfunctioning endogenous gene, or alternatively, a malfunctional version of a normally functioning endogenous gene. It can also be any other gene which expression is desired in a cell, depending on the purpose.

In some embodiments, the method of genome engineering may be particularly suitable for the transgenesis of an organism.

As used herein, the term “transgenesis” refers to the process of introducing one or several transgenes from one organism into another, with the intent of enabling the latter to transmit this transgene to its offspring.

The organism may be a plant or an animal. In some embodiments, the animal is not a human.

The skilled artisan is familiar with means and methods for transgenesis.

In some embodiments, the method comprises the steps of
transfecting or transducing an animal cell with the RNA molecule, or the non-viral vector or viral vector, in particular the retroviral vector, or the composition;
collecting the animal cell's nucleus;
inserting the animal cell's nucleus into an unfertilized oocyte;
allowing the oocyte to develop, thereby obtaining a transgenic animal, preferably wherein the transgenic animal is not a human.

In particular, the sequence of interest of the RNA molecule is a genome editor, and the nucleic acid sequence encoding the transgene of interest is brought to the cell in addition to the RNA molecule of the invention, for stable integration into the genome of said cell and development of a transgenic organism.

In some embodiments, the method of genome engineering may be particularly suitable for bioproduction (or recombinant production), in particular for the generation of stable expression systems.

Such method is applicable, for instance, to the production of recombinant proteins of interest in any suitable expression system, including without limitation, bacterial cells, yeast cells, insect cells, mammalian cells, and human cells.

The skilled artisan is familiar with means and methods for recombinant protein production.

In some embodiments, the method comprises the steps of
transfecting or transducing a cell or a population of cells with the RNA molecule, or the non-viral vector or viral vector, in particular the retroviral vector, or the composition;
culturing the cell or population of cells for a period of time sufficient to allow the production by said cell or population of cells of a protein of interest encoded by the RNA molecule;
recovering the protein of interest;
optionally, purifying the protein of interest.

In particular, the sequence of interest of the RNA molecule is a genome editor, and the nucleic acid sequence encoding the protein of interest is brought to the cell or population of cells in addition to the RNA molecule of the invention, for stable integration into the genome of said cell or population of cells and recombinant production of the protein of interest.

A fourth object of the invention is a nucleic acid system; a kit comprising said nucleic acid system; a cell or cell population comprising said nucleic acid system; and a method of producing an RNA virus vector, in particular a retroviral vector, as described above, using said nucleic acid system or said cell or cell population.

According to the invention, the nucleic acid system comprises:
(i) a first nucleic acid sequence encoding an RNA virus genome, in particular a retrovirus genome comprising at least a viral, preferably retroviral, gag and pol sequence; optionally wherein the pol sequence encodes a defective reverse-transcriptase (RT) or wherein the pol sequence lacks a RT gene;
(ii) a second nucleic acid sequence encoding an envelope glycoprotein; and
(iii) a third nucleic acid sequence encoding a recombinant expression cassette comprising, from 5′ to 3′:
   optionally, a 5′ long terminal repeat (LTR),
   a packaging sequence,
   optionally, a Rev-response element,
   an RNA Booster sequence characterized by a sequence mmsknkkkm,
   optionally, a sequence of interest,
   optionally, a post-transcriptional regulatory element, and
   optionally, a 3′ LTR;
      wherein the first and second nucleic acid sequences lack a functional packaging sequence.

Each nucleic acid sequence (i), (ii) and (iii) defined above is a DNA nucleic acid sequence. Hence, the RNA Booster sequence will comprise thymine in lieu of uracyl defined above for the RNA molecule. Exemplary RNA Booster sequences in the third nucleic acid sequence include thus, but are not limited to:
a deoxyribonucleic acid sequence mmskngkkm, preferably mmskngkgm, more preferably cmskhgkgm, even more preferably cmskwgkgm, yet even more preferably ccsuwgggm wherein:
   “m” indicates an adenine (a) or cytosine (c);
   “s” indicates a guanine (g) or a cytosine (c);
   “k” indicates a guanine (g) or a thymine (t);
   “h” indicates an adenine (a) or a cytosine (c) or a thymine (t);
   “w” indicates an adenine (a) or a thymine (t);
   “n” indicates any nucleotide;
      and in particular:
RNA Booster 1: cccgtgtgc
RNA Booster 2: aagtttggc
RNA Booster 3: ccctaggta
RNA Booster 4: ccgtggtgc
RNA Booster 5: aactggggc
RNA Booster 6: ccctcgggc
RNA Booster 7: cacgtgtgc
RNA Booster 8: cccttgggc
RNA Booster 9: ccgtaggga

In some embodiment, the first and second nucleic acid sequences are trans-complementation sequences, i.e., sequences encoding trans-complementation proteins or peptides which are intended to be part of the RNA viral vector, preferably the retroviral vector, as proteins or peptides, but not as nucleic acid information in its ribonucleic genome. These trans-complementation proteins or peptides are thus typically expressed in trans within a cell or population of cell during the production of the RNA viral vector, preferably the retroviral vector. In order to remain trans-complementary, trans-complementation sequences should be present in nucleic acid sequences lacking a functional packaging sequence.

In some embodiments, each nucleic acid sequence (i), (ii) and (iii) defined above may, independently from each other, be a linear nucleic acid or a plasmid.

The skilled artisan will readily understand that the first nucleic acid sequence encoding the RNA virus genome, preferably the retrovirus genome, can be provided in a single linear nucleic acid or plasmid, or in two or more linear nucleic acids or plasmids.

In some embodiments, the second nucleic acid sequence encodes an envelope glycoprotein, such as an envelope glycoprotein from (or derived from) an enveloped virus, possibly from a Retroviridae or from any other enveloped virus, such as, e.g., from a Rhabdoviridae, or a cellular glycoprotein or a synthetic glycoprotein.

In some preferred embodiments, the second nucleic acid sequence encodes an envelope glycoprotein from Indiana vesiculovirus (formerly known as vesicular stomatitis virus or VSV).

In some embodiments, the third nucleic acid sequence does not comprise a sequence of interest. If so, it is desirable however that the third nucleic acid sequence comprises at least one restriction site (and preferably at least two different restriction sites), or any other means suitable for introducing a gene of interest downstream the RNA Booster sequence.

According to the invention, the method of producing an RNA viral vector, preferably a retroviral vector, comprises the steps of:
1) transfecting a cell or cell population with the nucleic acid system as described above;
2) culturing the cell or cell population of a period of time sufficient for the production of the RNA viral vector, preferably the retroviral vector; and
3) recovering, and optionally purifying, the RNA viral vector, preferably the retroviral vector.

According to the invention, the method of producing a RNA viral vector, preferably a retroviral vector alternatively comprises the steps of:
1) providing a cell or cell population as described above;
2) culturing the cell or cell population of a period of time sufficient for the production of the RNA viral vector, preferably the retroviral vector; and
3) recovering, and optionally purifying, the RNA viral vector, preferably the retroviral vector.

According to the invention, the kit comprising the nucleic acid system as described above may comprise the first, second and third nucleic acid sequences in separate vials or containers.

Alternatively, two of the first, second and third nucleic acid sequences can be mixed in a single vial or container; in this case, the first and second nucleic acid sequences can preferably be mixed in a single vial or container, and the third nucleic acid sequence can be provided in a separate vial or container.

In some embodiments, the kit may further comprise instructions for use, in particular, instructions for performing the method of producing a RNA viral vector, preferably a retroviral vector, as described above.

### EXAMPLES

The present invention is further illustrated by the following examples.

### Materials and Methods

### Plasmids

The envelope trans-complementation plasmid encodes the vesicular stomatitis virus envelope glycoprotein (VSV-G) with SEQ ID NO: 1, under control of a cytomegalovirus-immediate early (CMV-IE) promoter.

The capsid trans-complementation plasmid encodes a functional integrase (with SEQ ID NO: 2) and a functional reverse transcriptase (with SEQ ID NO: 4) of HIV-1; or a mutant integrase with abolished integrase activity (SEQ ID NO: 2 comprising a D64V substitution, as set forth in SEQ ID NO: 3); or a mutant reverse transcriptase with abolished reverse transcriptase activity (SEQ ID NO: 4 comprising a D110E substitution, as set forth in SEQ ID NO: 5).

The vector plasmid encodes a recombinant expression cassette comprising a 5′ LTR (with SEQ ID NO: 6) and a 3′ LTR (with SEQ ID NO: 7) flanking a transgene (i.e., a sequence of interest), either GFP including a tobacco extension signal sequence (with SEQ ID NO: 8, encoding SEQ ID NO: 9), human fibroblast growth factor 9 (FGF9) (with SEQ ID NO: 10 encoding SEQ ID NO: 11), ovalbumin (with SEQ ID NO: 12 encoding SEQ ID NO: 13), or Cas9 (with SEQ ID NO: 14 encoding SEQ ID NO: 15), with or without a RNA Booster sequence in 5′ of the transgene (RNA Booster 1 to RNA Booster 10, according to Table 1) inserted in a SalI restriction site. Regulatory sequences such as retroviral psi packaging signal (with SEQ ID NO: 16), Rev-response element (RRE, with SEQ ID NO: 17) and WHV post-transcriptional regulatory element (WPRE, with SEQ ID NO: 18), were also included.

### Lentiviral Vector Production

Lentiviral vectors were generated by the transient transfection of 293T cells by using the calcium phosphate precipitation method. Briefly, cells were co-transfected with the VSV-G trans-complementation plasmid, the capsid trans-complementation plasmid and a vector plasmid. Supernatant was collected 48 hours after transfection, treated with DNaseI and filtered. Viral particles were then concentrated by ultracentrifugation and resuspended in 0.1 M PBS. The genome of particles was quantified for each stock by RT-qPCR to determine a titer of gRNA by μL.

### Cell Culture

293T cells were grown in Dulbecco's modified medium supplemented with antibiotics (100 U/mL penicillin and 100 mg/mL streptomycin) and 10% heat-inactivated fetal calf serum. The cells were plated and cultured in a humidified incubator at 37° C. in a 5% CO₂ and 90% air atmosphere.

Human PBMC were grown in RPMI-160+L-Glu medium supplemented with 1% HEPES 5 M, 0.1% P-mercaptoethanol 55 mM, antibiotics (100 U/mL penicillin and 100 mg/mL streptomycin) and 10% heat-inactivated fetal calf serum. The cells were plated and cultured in a humidified incubator at 37° C. in a 5% CO₂ and 90% air atmosphere.

Human dendritic cells were grown in RPMI-160+L-Glu medium supplemented with 1% HEPES 5 M, 100 ng/mL GM-CSF, 50 ng/mL IL-4, antibiotics (100 U/mL penicillin and 100 mg/mL streptomycin) and 10% heat-inactivated fetal calf serum. The cells were plated and cultured in a humidified incubator at 37° C. in a 5% CO₂ and 90% air atmosphere.

Human hematopoietic stem cells (HSC) were grown in StemSpan™ SFEM medium supplemented with the StemSpan™ CD34+ Expansion Supplement (10×). The cells were plated and cultured in a humidified incubator at 37° C. in a 5% CO₂ and 90% air atmosphere.

Human hair follicle dermal papilla cells were grown in HFDPC Basal culture medium with HFDPC supplement mix. The cells were plated and cultured in a humidified incubator at 37° C. in a 5% CO₂ and 90% air atmosphere.

GFP⁺ HeLa cells were generated with an integrating lentiviral vector expressing GFP. A clonal population with one integration and a stable expression of GFP was selected for the experiments. GFP⁺ HeLa cells were grown in Dulbecco's modified medium supplemented with antibiotics (100 U/mL penicillin and 100 mg/mL streptomycin) and 10% heat-inactivated fetal calf serum. The cells were plated and cultured in a humidified incubator at 37° C. in a 5% CO₂ and 90% air atmosphere.

### Transduction

293T cells were contacted and transduced with an RNA vector derived from lentivirus (with a mutated D110E reverse transcriptase) expressing GFP with one of RNA Booster 1 to 9, or without RNA Booster. During the transduction, the cells were incubated for 6 hours. GFP expression was measured 72 hours after transduction.

PBMC were contacted and transduced with an RNA vector derived from lentivirus (with a mutated D110E reverse transcriptase) expressing GFP with RNA Booster 8, or with an integrating lentiviral vector expressing GFP (without RNA Booster). GFP expression was measured by FACS 96 hours after transduction.

Human CD14⁺ monocytes were differentiated into dendritic cells during 4 days, before transduction, with GM-CSF (100 ng/mL) and IL-4 (50 ng/mL). Differentiation was checked by cytometry with an anti-CD1a antibody. Dendritic cells were then contacted and transduced with an RNA vector derived from lentivirus (with a mutated D110E reverse transcriptase) expressing GFP with RNA Booster 8, or with an integrating lentiviral vector expressing GFP (without RNA Booster), or with a non-integrating lentiviral vector expressing GFP (without RNA Booster). GFP expression was measured by FACS 96 hours after transduction.

Human hematopoietic stem cells (HSC) were contacted and transduced with an RNA vector derived from lentivirus (with a mutated D110E reverse transcriptase) expressing GFP with RNA Booster 8, or with an integrating lentiviral vector expressing GFP (without RNA Booster), or with a non-integrating lentiviral vector expressing GFP (without RNA Booster). GFP expression was measured by FACS 96 hours after transduction.

Human hair follicle dermal papilla cells were contacted and transduced with an RNA vector derived from lentivirus (with a mutated D110E reverse transcriptase) expressing FGF9 with RNA Booster 8. After transduction, the cells were treated with 300 nM of cortisol (which has a negative effect on proliferation). A control with or without VEGF (which inhibits the cortisol effect) was performed. Cell proliferation was measured through BrdU (5-bromo-2′-deoxyuridine) incorporation.

GFP⁺ HeLa cells were contacted and co-transduced with an RNA vector derived from lentivirus (with a mutated D110E reverse transcriptase) expressing Cas9 with RNA Booster 8 and a non-integrating lentiviral vector expressing a guide RNA targeting the GFP (without RNA Booster), or co-transduced with a non-integrating lentiviral vector expressing Cas9 (without RNA Booster) and a non-integrating lentiviral vector expressing a guide RNA targeting the GFP (without RNA Booster). GFP expression was measured by FACS 96 hours after.

### Flow Cytometry Analysis

GFP expression was analyzed by flow cytometry to determine the percentage of GFP-positive cells. Transduced cells were harvested, trypsinized, and fixed with 1% formaldehyde before analysis.

### Mouse Immunization

C57BL/6J mice were immunized with an RNA vector derived from lentivirus (with a mutated D110E reverse transcriptase) expressing ovalbumin with RNA Booster 8, or with a non-integrating lentiviral vector expressing ovalbumin (without RNA Booster). A boost injection with the same vector was administered 28 days after the prime injection. Injections were performed subcutaneously, intramuscularly, intranasally or intraperitoneally, with various combinations for the prime and boost injection. Immunization was measured through the dosage of ovalbumin-specific immunoglobulin G (IgG) 14 days after prime injection and again 14 days after boost injection.

Three controls were also carried out: a negative control, in which mice were administered saline; and two positive controls, in which mice were administered unadjuvanted ovalbumin or ovalbumin adjuvanted with alum.

### Ovalbumin-Specific IgG Dosage

About 0.15 mL of blood were taken from each animal into dry capillaries from the mandibular vessels under isoflurane gas anesthesia. The blood samples were kept at room temperature for at least 30 minutes and serum was prepared within 60 minutes of sampling by centrifugation for 10 minutes at 1500 g at 4° C.±2° C.). Serum was frozen within 120 minutes post-sampling and stored at −70° C.

Ovalbumin-specific IgG were measured from thawed blood samples using the “Mouse anti-OVA IgG antibody assay kit” (Chondrex, Inc.; Ref. 3011).

### Results

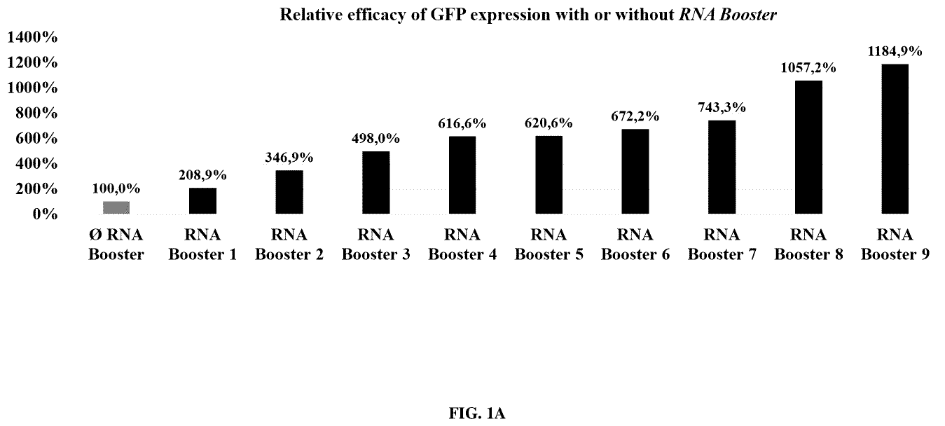

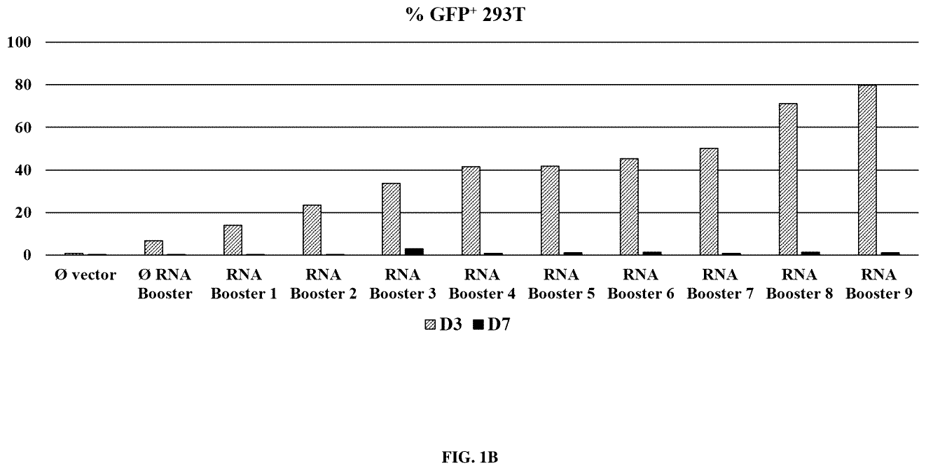

### GFP In Vitro Expression in 293T Cells (FIGS. 1A-B)

GFP expression from an RNA vector derived from lentivirus expressing GFP (with one of RNA Booster 1 to RNA Booster 9, or without RNA Booster) was compared at a same Multiplicity Of Infection (MOI).

As shown in FIG. 1A, the presence of any of RNA Booster 1-9 in 5′ of the transgene of interest in the RNA vector derived from lentivirus improved the efficacy of GFP expression in transduced 293T cells, as compared to a lentiviral vector without RNA Booster. The RNA Booster sequences were shown to enhance GFP expression by at least a factor 2 (for RNA Booster 1) and increasingly, up to a factor 12 (for RNA Booster 10).

As seen in FIG. 1B, GFP was well expressed in 293T cells at day 3 post-transduction, but not at day 7 post-transduction anymore, confirming the transientness of this expression system.

These data demonstrate show that presence of a RNA Booster as defined herein, in 5′ of a transgene of interest, is able to improve the efficacy of transient expression of this transgene of interest in 293T cells.

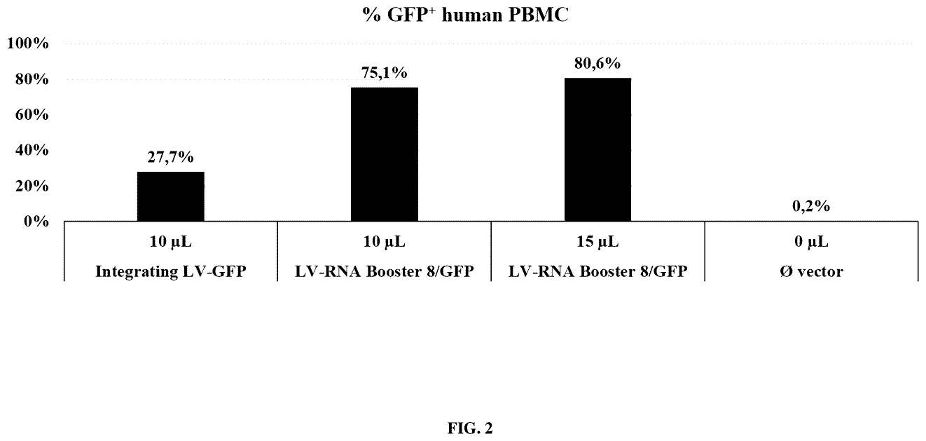

### GFP In Vitro Expression in Human PBMC (FIG. 2)

FIG. 2 shows that the RNA vector derived from lentivirus expressing GFP, in presence of RNA Booster 8, and both at 10 μL and 15 μL doses, enabled to transduce human PBMC with high efficacy and induced an increase of the GFP expression by a factor 2.7-2.9, as compared to the integrating vector without RNA Booster.

These data demonstrate that presence of a RNA Booster as defined herein, in 5′ of a transgene of interest, is able to improve the efficacy of expression of this transgene of interest in human PBMC.

This suggests that an RNA vector derived from lentivirus comprising such RNA Booster could be good candidates to induce transient expression of a gene of interest in PBMC, which cells are of interest in a wide range of immunotherapy strategies and gene therapy.

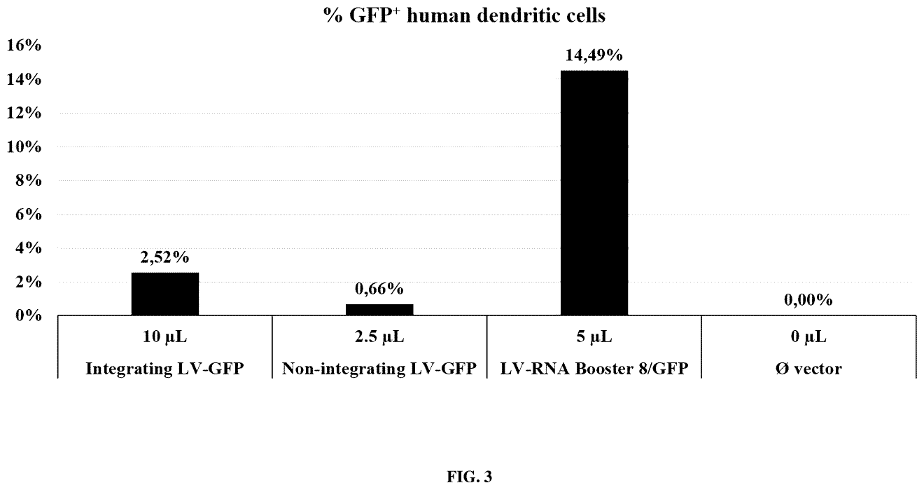

### GFP In Vitro Expression in Human Dendritic Cells (FIG. 3)

FIG. 3 shows that the RNA vector derived from lentivirus expressing GFP, in presence of RNA Booster 8, enabled to transduce human dendritic cells with high efficacy and induced an increase of the GFP expression 5.75 to 22 times higher than when using an integrative lentiviral vector or a non-integrative lentiviral vector in absence of RNA Booster, respectively.

This suggests that RNA vectors derived from lentivirus comprising such RNA Booster could be good candidates to induce transient expression of a gene of interest in primary cells such as dendritic cells.

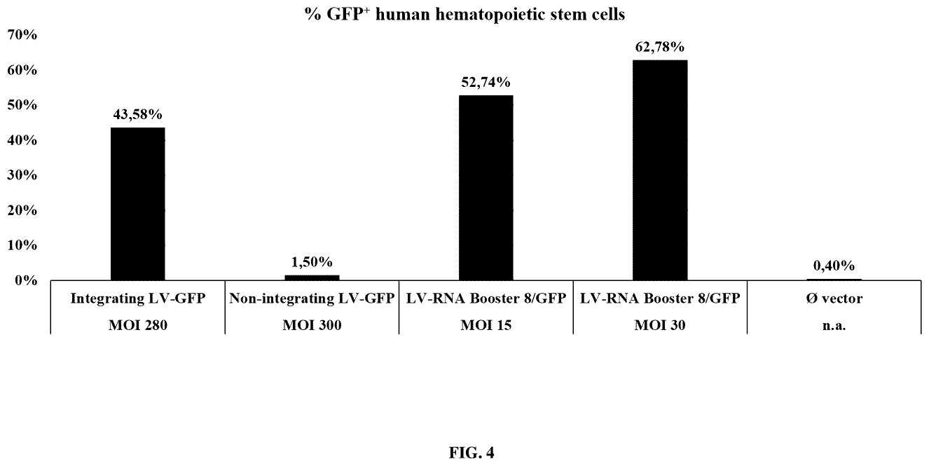

### GFP In Vitro Expression in Human Hematopoietic Stem Cells (FIG. 4)

FIG. 4 shows that the RNA vector derived from lentivirus expressing GFP, in presence of RNA Booster 8, enabled to transduce human hematopoietic stem cells with a higher efficacy than DNA integrating lentiviral vectors at a MOI 10 to 20 times lower, and induced an increase of the GFP expression around 35-42 times higher than when using a non-integrating lentiviral vector in absence of RNA Booster.

These data suggest that RNA vectors derived from lentivirus comprising such RNA Booster could be good candidates to induce transient expression of a gene of interest in human HSC, which cells are of interest in a wide range of immunotherapy strategies and gene therapy.

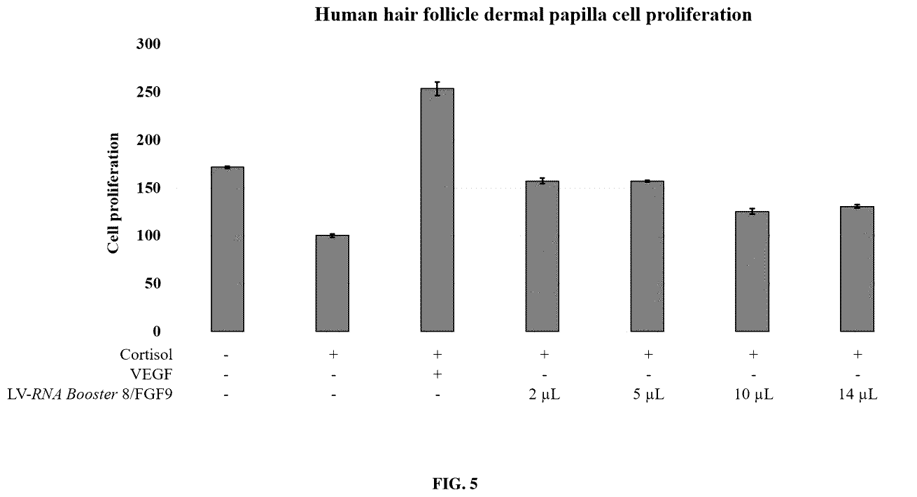

### In Vitro Proliferation Assay of Human Hair Follicle Dermal Papilla Cells (FIG. 5)

FIG. 5 shows that cortisol decreased cell proliferation, as compared to the non-treated condition (without cortisol, VEGF or vector), while adding VEGF and cortisol restored cell proliferation (with cortisol and VEGF, without vector). The RNA vector derived from lentivirus expressing FGF9, in presence of RNA Booster 8, enabled human hair follicle dermal papilla cells treated with cortisol to be transduced and to restore cell proliferation at all doses (2, 5, 10 and 14 μL), to levels similar to the non-treated control (without cortisol, VEGF or vector).

These data suggest that these RNA vectors derived from lentivirus comprising such RNA Booster could be useful for treating a variety of diseases or in non-therapeutic indications, for example in dermatology or cosmetology, to induce hair growth or promote skin healing.

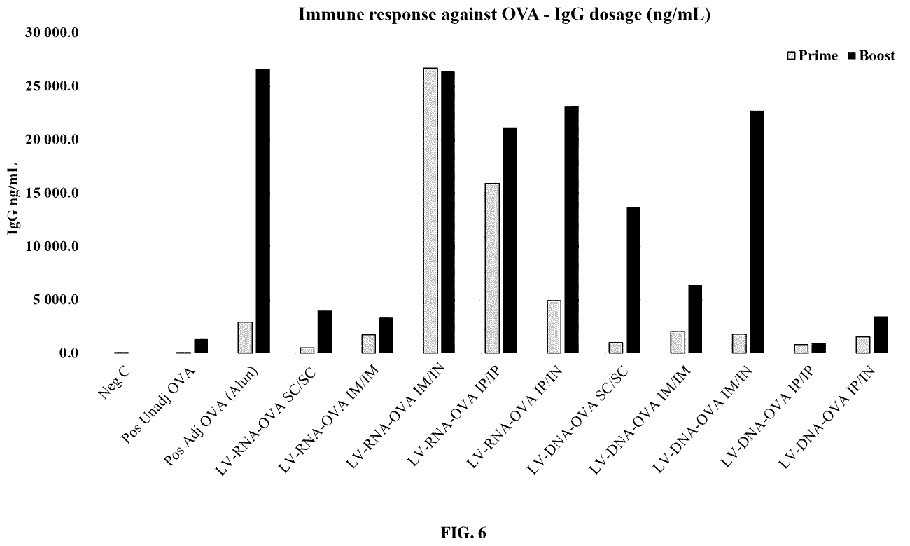

### In Vivo Vaccination Assay (FIG. 6)

FIG. 6 shows that an RNA vector derived from lentivirus expressing ovalbumin, in presence of RNA Booster 8, induced high levels of OVA-specific IgG when administered intramuscularly/intranasally, intraperitoneally/intraperitoneally, or intraperitoneally/intranasally (prime/boost—5×10⁸ transducing units (TU) per injection). In particular, this RNA vector derived from lentivirus induced higher levels of OVA-specific IgG as compared to a non-integrative lentiviral vector expressing ovalbumin administered by the same routes.

Interestingly, very high doses of OVA-specific IgG were obtained after an intramuscular prime injection of the RNA vector derived from lentivirus (and to a lesser extent, after an intraperitoneal prime injection), suggesting that a boost injection could not even be needed.

These data demonstrate that RNA vectors derived from lentivirus comprising such RNA Booster can improve immunization in mice, and suggest that these RNA vectors derived from lentivirus could be useful for vaccination.

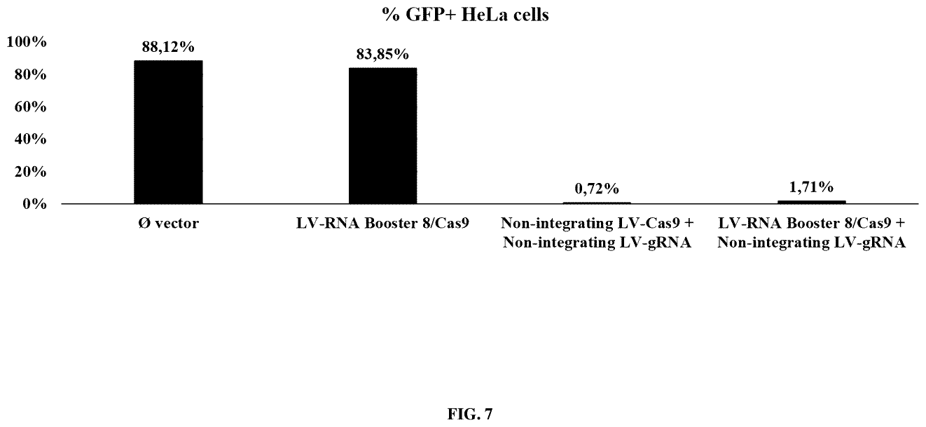

### Cas9 In Vitro Expression in HeLa Cells Constitutively Expressing GFP (FIG. 7)

FIG. 7 shows that an RNA vector derived from lentivirus expressing Cas9, in presence of RNA Booster 8, combined with a guide RNA targeting GFP brought to the cell using a non-integrating lentiviral vector, induces the knock-out of the GFP gene in HeLa cells with an efficacy close than 100%.

Although non-integrating lentiviral vectors expressing Cas9 and a gRNA yield similar results, using these vectors in ex vivo or in vivo therapy is not desirable since DNA molecules have a non-zero probability of recombination with another DNA molecule, such as with a DNA genome. This would induce adverse effects. Moreover, non-integrating lentiviral vectors have been shown in the art to exhibit a residual level of integration of 0.1-0.5%. Conversely, RNA vectors derived from lentivirus do not show any risk of reverse transcriptase activity leakage, which ensures thus 100% of information transfer in the form of RNA, which cannot recombine with DNA.

These data suggest that RNA vectors derived from lentivirus comprising such RNA Booster could be good candidates to induce transient expression of a genome editor, and could thus be useful for genome engineering in the field of bioproduction, cell therapy, gene therapy and transgenesis.

1. A method of treating skin and/or skin appendages in a subject in need thereof, comprising administering to the subject a ribonucleic acid (RNA) molecule comprising, from 5′ to 3′:
an RNA Booster sequence comprising or consisting of the following ribonucleic acid sequence: mmsknkkkm, wherein:
“m” indicates an adenine (a) or cytosine (c);
“s” indicates a guanine (g) or a cytosine (c);
“k” indicates a guanine (g) or a uracyl (u);
“n” indicates any nucleotide;
and
a sequence of interest.

2. The method according to claim 1, wherein treating skin and/or skin appendages includes one or several of inducing hair growth, preventing hair loss, inducing hair removal, hair coloring or bleaching, preventing hair graying, promoting hair thickening, promoting hair curling, prevention hair curling, promoting skin healing, preventing wrinkle formation, improving skin elasticity, inducing skin tone homogenization, and reducing sebum secretion.

3. The method according to claim 1, wherein the RNA Booster sequence comprises or consists of a ribonucleic acid sequence mmskngkkm, mmskngkgm, cmskhgkgm, cmskwgkgm, or ccsuwgggm, wherein:
“m” indicates an adenine (a) or cytosine (c);
“s” indicates a guanine (g) or a cytosine (c);
“k” indicates a guanine (g) or a uracyl (u);
“h” indicates an adenine (a) or a cytosine (c) or a uracyl (u);
“w” indicates an adenine (a) or a uracyl (u);
“n” indicates any nucleotide.

4. The method according to claim 1, wherein the RNA Booster sequence is selected from the group consisting of:
RNA Booster 9 comprising or consisting of the sequence ccguaggga;
RNA Booster 8 comprising or consisting of the sequence cccuugggc;
RNA Booster 7 comprising or consisting of the sequence cacgugugc;
RNA Booster 6 comprising or consisting of the sequence cccucgggc;
RNA Booster 5 comprising or consisting of the sequence aacuggggc;
RNA Booster 4 comprising or consisting of the sequence ccguggugc;
RNA Booster 3 comprising or consisting of the sequence cccuaggua;
RNA Booster 2 comprising or consisting of the sequence aaguuuggc; and
RNA Booster 1 comprising or consisting of the sequence cccgugugc.

5. The method according to claim 1, wherein said RNA molecule is comprised within a non-viral vector.

6. The method according to claim 1, wherein said RNA molecule is packaged into an RNA virus vector derived from a Group III, Group IV, Group V or Group VI RNA virus.

7. The method according to claim 6, wherein the Group VI RNA virus is a Retroviridae.

8. The method according to claim 7, wherein the Retroviridae is a lentivirus.

9. The method according to claim 8, wherein the lentivirus is selected from the group consisting of human immunodeficiency viruses, simian immunodeficiency virus (SIV), feline immunodeficiency virus (FIV), bovine immunodeficiency virus (BIV), puma lentivirus (PLV), equine infectious anemia virus (EIAV), caprine arthritis encephalitis virus (CAEV), Visna-maedi virus, and Jembrana disease virus.

10. The method according to claim 8, wherein the lentivirus is a human immunodeficiency virus (HIV).

11. The method according to claim 6, wherein the Group VI RNA virus vector is reverse transcriptase (RT)-defective.

12. The method according to claim 6, wherein the RNA molecule further comprises one or several of:
a 5′ long terminal repeat (LTR),
a packaging sequence,
a Rev-response element sequence,
a post-transcriptional regulation element sequence, and
a 3′ LTR.

13. The method according to claim 1, wherein the sequence of interest is selected from the group consisting of sequences encoding the glia-activating factor (FGF9), the hepatocyte growth factor (HGF), the platelet-derived growth factor (PDGF), the fibroblast growth factor 5 (FGF5), the fibroblast growth factor 10 (FGF10), the transforming growth factor β1 (TGFβ1), the transforming growth factor α (TGFα), the keratinocyte growth factor (KGF), the insulin-like growth factor 1 (IGF-1), the insulin-like growth factor 2 (IGF-2), the insulin-like growth factor-binding protein 5 (IGFBP5), the vascular endothelial growth factor (VEGF), the acidic fibroblast growth factor (aFGF), the basic fibroblast growth factor (bFGF), the epidermal growth factor (EGF), the L-dopachrome tautomerase (TRP2), noggin (NOG), protaetiamycine, thioredoxin (TRX), superoxide dismutase (SOD1), the stem cell factor (SCF), and the human growth hormone (hGH).

14. The method according to claim 1, wherein the sequence of interest encodes the glia-activating factor (FGF9).

15. The method according to claim 1, wherein the method is for inducing hair growth and/or promoting skin healing.

16. The method according to claim 14, wherein the method is for inducing hair growth and/or promoting skin healing.

17. The method according to claim 1, wherein the RNA molecule is administered to the subject in need thereof topically.

18. The method according to claim 17, wherein the RNA molecule is administered to the subject in need thereof cutaneously or transdermally

19. The method according to claim 18, wherein the RNA molecule is administered to the subject in need thereof transdermally after collagen induction therapy (CIT) or through a transdermal patch.
